(19) United States
(12) Patent Application Publication
(10) Pub. No.: US 2022/0274973 A1
(43) Pub. Date: Sep. 1, 2022

(54) Methods And Compositions For Treating Sickle Cell Disease With A Ferroportin Inhibitor (VIT-2763)

(71) Applicant: Vifor (International) AG, St. Gallen (CH)

(72) Inventors: Vania Manolova, St. Gallen (CH); Franz Dürrenberger, St. Gallen (CH); Naja Nyffenegger, St. Gallen (CH)

(21) Appl. No.: 17/636,575

(22) PCT Filed: Oct. 22, 2020
(86) PCT No.: PCT/EP2020/079802

§ 371 Date: Feb. 18, 2022

Related U.S. Application Data
(60) Provisional application No. 62/924,556, filed on Oct. 22, 2019.

(30) Foreign Application Priority Data
Oct. 22, 2019 (EP) 19000483.8
Mar. 17, 2020 (EP) 20163777.4
May 25, 2020 (EP) 20176336.4

(51) Int. Cl. C07D 413/14; A61P 7/00; A61P 29/00; A61P 1/16; A61P 13/12

(52) U.S. Cl. CPC C07D 413/14; A61P 7/00; A61P 29/00; A61P 1/16; A61P 13/12

(57) ABSTRACT

The invention relates to compounds of the formula

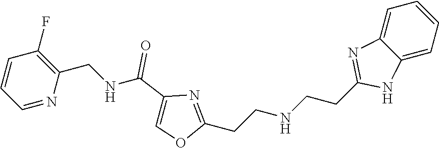 [SMILES: O=C(NCc1ncccc1F)c1coc(CCNCCc2nc3ccccc3[nH]2)n1]

and pharmaceutically acceptable salts thereof for the use in the treatment of sickle cell disease and for the use in preventing and treating vascular inflammation and vaso-occlusion.

### BACKGROUND

Sickle cell disease (SCD) is an inherited disorder of hemoglobin synthesis characterized by life-long severe hemolytic anemia, recurrent pain crisis, chronic organ system damage and a marked decrease in life expectancy. SCD results from a mutation in the hemoglobin (Hb) β gene that causes an amino acid substitution in the β globin protein and results in the sickle hemoglobin (HbS) allele βS. In the mouse model of SCD (Townes mice) the murine Hb genes have been deleted and replaced by the human sickle Hb gene. Mice homozygous for HbS express exclusively human sickle Hb and develop pathologies closely resembling SCD in humans with rigid sickle-shaped red blood cells (sRBCs), hemolytic anemia, iron overload, expansion of splenic red pulp, inflammation, increased adhesion of blood cells to endothelial vasculature leading to vaso-occlusion/vascular occlusion (VO) and organ injuries. Polymerization of deoxy-HbS shortens the lifespan of sickle RBCs and promotes intravascular and extravascular hemolysis. The intravascular hemolysis leads to release of cell-free Hb from RBCs. The extracellular Hb is easily oxidized from ferrous (Fe²⁺) to ferric (Fe³⁺) Hb (metHb), from which heme readily dissociates into the vasculature leading to oxidative stress, inflammation, VO, ischemia and tissue injury (Umbreit J., Am. J. Hematol., 2007).

Conventional (approved) drugs for treating SCD symptoms associated therewith are Hydroxyurea (Droxia™, Hydrea™, Siklos™), L-glutamine oral powder (Endari™) Crizanlizumab (Adakveo™), and Voxelotor (Oxbryta™)

Daily administration of hydroxyurea reduces the frequency of painful crises and might reduce the need for blood transfusions and hospitalizations. It may increase risk of infections.

L-glutamine oral powder (Endari) helps in reducing the frequency of pain crises.

Crizanlizumab (Adakveo), an intraveneously administered drug, helps to reduce the frequency of pain crises. Side effects can include nausea, joint pain, back pain and fever.

Voxelotor (Oxbryta), an orally administered drug improves anemia in patients with sickle cell disease. Side effects can include headache, nausea, diarrhea, fatigue, rash and fever.

Further, pain-relieving medications are regularly administered to SCD patients, which help to relieve pain during sickle cell pain crises, however, without treating the cause of the pain.

### OBJECT OF THE INVENTION

It was an object of the present invention to provide a novel, improved drug or therapy for treating SCD. In particular, the novel drug or SCD therapy should improve, alleviate or change one or more of the markers, conditions or events relevant with SCD, and as defined further herein, towards a normalized level. A further object of the invention was to provide a SCD drug or therapy with improved safety profile compared to conventional hydroxyurea treatment. A further object of the invention was to provide a SCD drug or therapy with at least the same or even improved safety profile compared to conventional voxelotor treatment. The present invention aims at providing an improved SCD therapy, considering one or more of the aspects discussed herein in more detail below.

### SUMMARY AND DESCRIPTION OF THE INVENTION

Described herein are methods for treating SCD comprising administering a compound of the following formula (Compound 127)

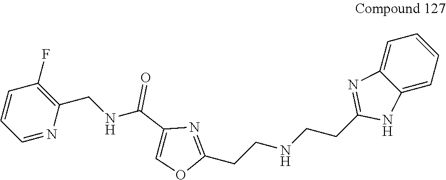 [SMILES: O=C(NCc1ncccc1F)c1coc(CCNCCc2nc3ccccc3[nH]2)n1]

or a pharmaceutically acceptable salt thereof. Among the suitable salts are: benzoic acid salt, HCl salt, citric acid salt, fumaric acid salt, lactic acid salt, malic acid salt, maleic salt, methanesulfonic acid salt, phosphoric acid salt, succinic acid salt, sulfuric acid salts, tartaric acid salts and toluenensufonic acid salts. In various embodiments, the ratio of compound to salt is 1:1, 2:1, 1:2 or 1:3. As used herein, a salt of a compound refers to any ratio of compound to salt unless a specific ratio is indicated.

Compound 127 and methods for synthesizing Compound 127 are described in WO2017/068089 and WO2017/068090A1, hereby incorporated by reference. Specific salts of compound 127, as well as a variety of polymorphs of Compound 127 are described in WO 2018/192973, hereby incorporated by reference. The potential use of the specific salts disclosed therein in treating sickle cell disease is generally mentioned in a list among various other indications. Example 13 describes single dose intravenous and oral pharmacokinetic studies with H₂SO₄ and HCl-mono salts of compound 127. Vania Manolova: “First-in-class oral Ferroportin Inhibitor: Mode of Action and Efficacy in a mouse model of Beta-Thalassemia Intermedia”; EHA Abstract, 14.06.2019 discloses to use compound 127 in the treatment of thalassemia intermedia but remains silent about any specific salt form thereof. Further, a potential efficacy in the treatment of sickle cell disease is also not mentioned therein.

J. H. Baek et al. “Ferroportin inhibition attenuates plasma iron, oxidant stress, and renal injury following red blood cell transfusion in guinea pigs”; TRANSFUSION, 00, 1-11, 2020 report results in the attenuation of plasma iron, NTBI levels, oxidative stress and cellular injury by intravenously administering the small-molecule ferroportin inhibitor VIT-2653, provided by Vifor (International) Ltd., immediately after acute red blood cell transfusions in a model with guinea pigs.

The ferroportin-hepcidin axis regulates blood iron levels. Compound 127 competes with hepcidin for ferroportin binding and internalization. Compound 127 inhibits ferroportin and thereby blocks iron transport to blood.

The term “treat”, “treatment” or “treating” in the context of the new use of the present invention includes amelioration of at least one symptom of or pathological condition associated with SCD. The term “treat”, “treatment” or “treating” in the context of the present invention further includes prophylaxis. The treatment with Compound 127 according to the present invention in particular improves, alleviates or changes one or more of the following markers, conditions or events, e.g. towards a normalized level.

### EFFECTS OF THE TREATMENT ACCORDING TO THE INVENTION

A particular aspect of the invention relates to the Compound 127 as described anywhere herein for the use in the treatment, prevention or alleviation of one or more of the markers, conditions or events describes supra or infra, or in particular in the Examples.

In some cases, treatment of a subject suffering from SCD with Compound 127 decreases hemolysis (e.g., as assessed by a decrease in cell-free hemoglobin (Hb), a decrease in cell-free heme, a decrease in total and indirect plasma bilirubin or a decrease in serum LDH (lactate dehydrogenase).

In some cases, treatment of a subject suffering from SCD with Compound 127 improves one or more of total serum iron, serum ferritin, serum transferrin, and calculated TSAT (transferrin saturation).

In some cases, treatment of a subject suffering from SCD with Compound 127 decreases reticulocytosis, improves reticulocyte counts and/or % reticulocytes.

In some cases, treatment of a subject suffering from SCD with Compound 127 decreases one or more of total Hb, RBC counts, hematocrit, mean corpuscular volume (MCV), mean corpuscular hemoglobin (MCH), and corpuscular haemoglobin concentration mean (CHCM).

In some cases, treatment of a subject suffering from SCD with Compound 127 improves one or more of RBC distribution width (RDW), platelet and reticulocyte counts.

In some cases, treatment of a subject suffering from SCD with Compound 127 improves microcytic RBC (RBC volume versus Hb scatterplot).

In some cases, treatment of a subject suffering from SCD with Compound 127 achieves changes in abnormal RBCs (sickling) and/or improves RBC sickling (peripheral blood smear).

In some cases, treatment of a subject suffering from SCD with Compound 127 decreases leukocytosis.

In some cases, treatment of a subject suffering from SCD with Compound 127 decreases blood leukocyte counts (e.g., decreases blood neutrophil counts and/or blood lymphocyte counts).

In some cases, treatment of a subject suffering from SCD with Compound 127 decreases extravascular and/or intravascular hemolysis.

In some cases, treatment of a subject suffering from SCD with Compound 127 improves one or more haemeoloysis markers, such as indirect/total bilirubin, blood inflammatory markers as measured by hsCRP (high sensitivity C-reactive protein), IL-1 and IL-6 (interleukin), TNF-alpha, sVCAM-1, endothelin-1, sP-selectin, sICAM-1, and xanthine oxidase.

In some cases, treatment of a subject suffering from SCD with Compound 127 improves one or more of RBC indices, including Hb concentration, RBC count, haematocrit (Hct), mean corpuscular volume (MCV), mean corpuscular Hb (MCH), mean corpuscular Hb concentration (MCHC), corpuscular Hb concentration mean (CHCM), RBC distribution width, platelet and reticulocyte counts, % reticulocytes, % hypochromic, microcytic RBC (RBC volume versus Hb scatterplot), CHCM (corpuscular haemoglobin concentration mean), total serum iron, serum ferritin, serum transferrin, calculated TSAT, hepcidin, EPO (erythropoietin), NTBI (non-transferrin bound iron), soluble transferrin receptor (sTFR), (sTFR-2), and LDH.

This means, one or more of the parameters mentioned above and below can be determined to evaluate the efficacy of the compound of the present invention in treatment of SCD. The Compound 127 of the present invention is suitable to improve at least one of these parameters.

In the sense of the present invention the term “improves” or “improvement” may cover a modulation or change of the respective marker or condition in the sense of a therapeutic effect.

More particularly, the treatment of SCD according to the present invention may result in:

Reduced NTBI levels in a patient by at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or at least 100%, determined at any time point within a time period of up to 72 hours, up to 60 hours, up to 48 hours, up to 36 hours, up to 24 hours, or up to 12, 8, 6, 5, 4, 3, 2, 1 and 0.5 hours following the administration and as compared to the NTBI levels in the patient determined at any time point within 0.5, 1, 2, 3, 4, 5, 6, 8, 12, 24, 36, or 48 hours, or up to <1 week prior to the commencement of treatment of the invention.

Reduced LPI levels in a patient by at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or at least 100%, determined at any time point within a time period of up to 72 hours, up to 60 hours, up to 48 hours, up to 36 hours, up to 24 hours, or up to 12, 8, 6, 5, 4, 3, 2, 1 and 0.5 hours following the administration and as compared to the total LPI levels in the patient determined at any time point within 0.5, 1, 2, 3, 4, 5, 6, 8, 12, 24, 36, or 48 hours, or up to <1 week prior to the commencement of treatment of the invention.

Improvement of at least one of the parameters Hct, MCV, MCH, RDW and reticulocyte numbers in the patient by at least 5%, 10%, 15%, 20%, 25%, 30%, 35%, 40%, 45%, 50%, 55%, 60%, 65%, 70%, 75%, 80%, 85%, 90%, 95% or at least 100%, determined at any time point within a time period of up to one week, up to 2 weeks, up to 3 weeks, up to 4 weeks, up to 3 months following the first administration and as compared to the respective parameter in the subject determined at any time point within 1 week, 2 weeks, 3 weeks, or 4 weeks prior to the commencement of treatment of the invention.

Reduced serum ferritin levels in the patient by at least 5%, 10%, 15%, 20%, 25%, 30%, 35%, 40%, 45%, 50%, 55%, 60%, 65%, 70%, 75%, 80%, 85%, 90%, 95% or at least 100%, determined at any time point within a time period of up to one week, up to 2 weeks, up to 3 weeks, up to 4 weeks, up to 3 months following the first administration and as compared to the serum ferritin levels in the patient determined at any time point within 1 week, 2 weeks, 3 weeks, or 4 weeks prior to the commencement of treatment of the invention.

In some cases, treatment of a subject suffering from SCD with Compound 127 reduces or prevents further iron accumulation in the liver, kidney and/or spleen.

Accordingly, in a further aspect, the new treatment may result in a decrease in liver, kidney and/or spleen iron concentration in the SCD patient by at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or at least 100%, determined at any time point within a time period of up to one week, up to 2 weeks, up to 3 weeks, up to 4 weeks, up to 3 months following the first administration and as compared to the levels of liver, kidney and/or spleen iron concentration in the SCD patient determined at any time point within 1 week, 2 weeks, 3 weeks, or 4 weeks prior to the commencement of treatment of the invention.

In some case, treatment with Compound 127 decreases iron overload in kidney.

In some cases, treatment of a subject suffering from SCD with Compound 127 reduces vascular inflammation markers, e.g. the levels of sVCAM-1.

In some cases, treatment of a subject suffering from SCD with Compound 127 reduces vascular inflammation.

In some cases, treatment of a subject suffering from SCD with Compound 127 reduces the blood cell adhesion in inflamed venules or to microvasculature and improves the blood flow in microvessels.

In some cases, treatment of a subject suffering from SCD with Compound 127 reduces blood cell adhesion to microvasculature, vaso-occlusion (VO) and alleviates VO events.

In some cases, treatment of a subject suffering from SCD with Compound 127 reduces the frequency of VOC or painful VOC crises and/or prevents recurrent painful VOC including ACS (Acute chest syndrome).

In some cases, treatment of a subject suffering from SCD with Compound 127 reduces the frequency of VOC or painful VOC crises and/or prevents recurrent painful VOC including ACS in the patient within 1 week, 2 weeks, 3 weeks or 4 weeks, 2 months, 3 months, 4 months, 6 months, 8 months, 9 months, 12 months, 24 months, prior to the commencement of treatment of the invention; or achieving that the SCD patient does not suffer from VOC or painful VOC crises for at least 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 5 months, 6 months, 7 months, 8 months, 9 months, 10 months, 11 months, 12 months, 18 months, 24 months or even longer after treatment.

In some cases, treatment of a subject suffering from SCD with Compound 127 achieves a reduction in mean number of painful (VOC) crises through 48 weeks.

In some cases, treatment of a subject suffering from SCD with Compound 127 achieves a reduction in mean number of painful (VOC) crises through 48 weeks in OH-Urea naïve patients.

A preferred aspect relates to the Compound 127 as described anywhere herein for the use in the treatment, prevention or alleviation of vascular inflammation or VO and VO events.

In some cases, treatment of a subject suffering from SCD with Compound 127 reduces the need for RBC transfusions, such as in particular reduction of transfusion burden in the patient compared to the transfusion burden for the patient within 1 week, 2 weeks, 3 weeks or 4 weeks, 2 months, 3 months, 4 months, 6 months, 8 months, 9 months, 12 months, 24 months, prior to the commencement of treatment of the invention; or achieving that the SCD patient does not require red blood cell transfusion for at least 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 5 months, 6 months, 7 months, 8 months, 9 months, 10 months, 11 months, 12 months, 18 months, 24 months or even longer up to independence from red blood cell transfusions after treatment.

In a further aspect, the new treatment may result in an improvement in the quality of life in the SCD patients as compared to the quality of life in the SCD patients determined within the 1, 2, 3, or 4 week(s) prior to the commencement of treatment of the invention. The improvement of Quality of life is determined within 3, 6, 9, 12, 15, 18, 21 or 24 months after the commencement of the treatment. Quality of Life can be determined by evaluating change in patient reported outcomes (PRO) using Adult Sickle Cell Quality-of-Life Measurement System (ASCQ-ME).

The determination of the above defined parameters can be carried out using conventional methods of the art, in particular by those described in the unpublished PCT-application PCT/EP2020/070391, the respective content thereof being herewith enclosed by reference.

### Patient Groups to be Treated

In principle, the subjects to be treated in the new use according to the invention can be any mammals such as rodents and primates, and in a preferred aspect the new medical use relates to the treatment of humans. The subjects suffering from SCD and to be treated with the new method according to the invention are also designated as “patients”.

The subjects to be treated can be of any age. A preferred aspect of the invention relates to the treatment of children and adolescents. Accordingly, in a preferred aspect of the invention the subjects to be treated with the new methods described herein are 18 years old. More particularly, the subjects to be treated with the new methods described herein are ≤16 years old, ≤15 years old, ≤14 years old, ≤13 years old, ≤12 years old, ≤11 years old, ≤10 years old, ≤9 years old, ≤8 years old, ≤7 years old, ≤6 years old, or ≤5 years old. In a further aspect of the invention the subjects to be treated with the new methods described herein are 1-3 years old, 3-5 years old, 5-7 years old, 7-9 years old, 9-11 years old, 11-13 years old, 13-15 years old, 15-20 years old, 20-25 years old, 25-30 years old, or >30 years old. Preferably pediatric patients to be treated are ≤16 years old or ≤12 years old. In the case of treating children, it is preferred that the subjects to be treated with the new methods described herein are ≥2 years old, preferably ≥2 years old and ≤16 years old or ≥12 years old.

In a further aspect of treating adults and adolescents, the patients are ≥12 years old or ≥16 years old.

In the case of treating adults, the subjects to be treated with the new methods described herein are preferably 18 to 50 years old, preferably 18-25 years old, 20-25 years old, 25-30 years old, 30-35 years old, 35-40 years old, 40-45 years old, 45-50 years old. It is also possible to treat elder adults being 50-55 years old, 55-60 years old, or greater than 60 years old. In the case of treating elderly patients the subjects to be treated with the new methods described herein are 60 to 80 years ols, such as 60-65 years old, 65-70 years old, 70-75 years old, 75-80 years old, or greater than 80 years old.

The treatment of children and adolescents is particularly preferred due to the significant advantages provided by the treatment with the compounds of the present invention. Said compounds can be administered orally, which is advantageous over parenteral administration. Further, the orally bioavailable compound of the present invention turned out to have a moderate bioavailability and half-life in the body and is thus relatively quickly washed out. This leads to less adverse effects and a faster reversibility of the drug, which is of particular importance in the treatment of children.

Compound 127 can be used to treat patients suffering from various forms of SCD, including: HbSS, HbSC, HbSO thalassemia, HbSβ+ thalassemia, HbSD, HbSE, and HbSO. In particular, Compound 127 can be used to treat patients suffering from HbSS or HbSO thalassemia.

Compound 127 can particularly be used to treat patients suffering from SCD, as defined anywhere herein, being inadequately controlled on monotherapy, e.g. with Hydroxyurea.

Compound 127 can be used to treat patients suffering from SCD, as described herein, having one or more VOC per year.

Compound 127 can be used to treat patients suffering from SCD, as described herein, having one or more and no more than 6 VOC per year.

Compound 127 can be used to treat patients suffering from SCD, as described herein, with an absolute reticulocyte count and % reticulocyte count of >1.5 ×upper limit of normal (ULN).

Compound 127 can be used to treat patients having a history of partial or total splenectomy, having any history or clinically important finding of cardiac or pulmonary disorders, and/or having received or receiving frequently or regularly red blood cell (RBC) transfusion therapy (including chronic, prophylactic, or preventive transfusion to treat SCD).

### Administration Forms

The compounds according to the invention are preferably provided in medicaments or pharmaceutical compositions in the form of oral administration forms, including e.g. pills, tablets, such as enteric-coated tablets, film tablets and layer tablets, sustained release formulations for oral administration, depot formulations, dragees, granulates, emulsions, dispersions, microcapsules, microformulations, nanoformulations, liposomal formulations, capsules, such as enteric-coated capsules, powders, microcrystalline formulations, epipastics, drops, ampoules, solutions and suspensions for oral administration.

In a preferred embodiment of the invention the compounds according to the invention are administered in the form of a tablet or capsule, as defined above. More preferred is a capsule filled with the drug Compound 127. These may be present, for example, as acid resistant forms or with pH dependent coatings.

The drug compound may be filled into capsules as the pure drug substance or in the form of a pharmaceutical composition comprising further pharmaceutically acceptable adjuvants, auxiliaries, solvents, additives etc..

Generally, the administration forms comprising the compound of the present invention may comprise further pharmaceutically acceptable adjuvants, auxiliaries, fillers, solvents, additives etc..

A said pharmaceutical composition may contain, for example up to 99 weight-% or up to 90 weight-% or up to 80 weight-% or or up to 70 weight-% of the drug compound of the present invention, the remainder being each formed by pharmacologically acceptable carriers and/or auxiliaries and/or solvents and/or optionally further pharmaceutically active compounds.

The pharmaceutically acceptable carriers, auxiliary substances or solvents etc. are common pharmaceutical carriers, auxiliary substances or solvents, including various organic or inorganic carrier and/or auxiliary materials as they are customarily used for pharmaceutical purposes, in particular for solid medicament formulations. Examples include excipients, such as saccharose, starch, mannitol, sorbitol, lactose, glucose, cellulose, talcum, calcium phosphate, calcium carbonate; binding agents, such as cellulose, methylcellulose, hydroxypropylcellulose, polypropyl pyrrolidone, gelatine, gum arabic, polyethylene glycol, saccharose, starch; disintegrating agents, such as starch, hydrolyzed starch, carboxymethylcellulose, calcium salt of carboxymethylcellulose, hydroxypropyl starch, sodium glycol starch, sodium bicarbonate, calcium phosphate, calcium citrate; lubricants, such as magnesium stearate, talcum, sodium laurylsulfate; flavorants, such as citric acid, menthol, glycin, orange powder; preserving agents, such as sodium benzoate, sodium bisulfite, paraben (for example methylparaben, ethylparaben, propylparaben, butylparaben); stabilizers, such as citric acid, sodium citrate, acetic acid and multicarboxylic acids from the titriplex series, such as, for example, diethylenetriaminepentaacetic acid (DTPA); suspending agents, such as methylcellulose, polyvinyl pyrrolidone, aluminum stearate; dispersing agents; diluting agents, such as water, organic solvents; waxes, fats and oils, such as beeswax, cocoa butter; polyethylene glycol; white petrolatum; etc.

Liquid medicament formulations, such as solutions, suspensions and gels usually contain liquid carrier, such as water and/or pharmaceutically acceptable organic solvents. Furthermore, such liquid formulations can also contain pH-adjusting agents, emulsifiers or dispersing agents, buffering agents, preserving agents, wetting agents, gelatinizing agents (for example methylcellulose), dyes and/or flavouring agents, for example as defined above. The compositions may be isotonic, that is, they can have the same osmotic pressure as blood. The isotonicity of the composition can be adjusted by using sodium chloride and other pharmaceutically acceptable agents, such as, for example, dextrose, maltose, boric acid, sodium tartrate, propylene glycol and other inorganic or organic soluble substances. The viscosity of the liquid compositions can be adjusted by means of a pharmaceutically acceptable thickening agent, such as methylcellulose. Other suitable thickening agents include, for example, xanthan gum, carboxymethylcellulose, hydroxypropylcellulose, carbomer and the like. The preferred concentration of the thickening agent will depend on the agent selected.

Pharmaceutically acceptable preserving agents can be used in order to increase the storage life of the liquid composition. Benzyl alcohol can be suitable, even though a plurality of preserving agents including, for example, paraben, thimerosal, chlorobutanol and benzalkonium chloride can also be used.

Accordingly, a further aspect of the present invention relates to the compounds according to the invention, including pharmaceutically acceptable salts, solvates, hydrates and polymorphs thereof, as well as medicaments, compositions and combined preparations comprising the same for the use in the treatment of SCD as defined herein in the form of oral administration forms.

### Dosing Regimen

The compounds according to the invention for the use according to the present invention can be administered by one of the following dosing regimens:

In one aspect the compounds according to the invention can be administered to a patient in need thereof in a dose of 0.001 to 500 mg, for example 1 to 4 times a day, preferably once or twice daily. However, the dose can be increased or reduced depending on the age, body weight, condition of the patient, severity of the disease or type of administration. In a further aspect of the invention the compounds of the invention can be administered as a dose of 0.1 mg, 0.2 mg, 0.3 mg, 0.4 mg, 0.5 mg, 0.6 mg, 0.7 mg, 0.8 mg, 0.9 mg, 1 mg, 1.5 mg, 2 mg, 2.5 mg, 3 mg, 3.5 mg, 4 mg, 4.5 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 325 mg, 350 mg, 375 mg, 400 mg, 425 mg, 450 mg, 475 mg, 500 mg.

Preferred is a dose of between 0.5 to 500 mg, more preferred between 1 to 300 mg or 3 to 300 mg, more preferred between 1 to 250 mg or 5 to 250 mg.

Most preferred is a dose of 5 mg, 15 mg, 30 mg, 60 mg, 120 mg or 240 mg. Particularly preferred are doses of 30 mg, 60 mg, 90 mg, 120 mg or 240 mg, more particularly of 30 mg, 60 mg, 90 mg or 120 mg. Most preferred are doses of 30 mg, 60 mg and 120 mg.

It is possible to administer the above defined dosages as a total daily dose either in a single dose daily or divided into sub-doses for administration twice or more times daily.

In a further preferred aspect a 30 mg, 60 mg, 90 mg or 120 mg daily dose is preferred which is administered as a single dose once daily. In a further aspect a 60 mg or 120 mg daily dose is administered as two 30 mg doses or as two 60 mg doses, respectively, twice daily. It is also possible to administer a 90 mg daily dose as three 30 mg doses, three times daily.

In a further aspect a dose between 0.001 to 60 mg/kg body weight, between 0.01 to 60 mg/kg body weight, between 0.1 to 60 mg/kg body weight, or between 0.5, 1, 2, 3, 4, 5, 6, 7, 8, 9, 10, 15, 20, 25, 30, 35 and up to 60 mg/kg body weight can be administered. It is possible to administer a dose of 120 mg or up to 240 mg for patients with ≥50 kg body weight and of 60 mg for patients with ≥50 kg body weight, in each case once or twice daily. It is preferred to administer a dose of 60 mg for patients with ≥50 kg body weight and of 30 mg for patients with ≤50 kg body weight, in each case once or twice daily. It is further preferred to administer the above defined doses of 30 mg and 60 mg to patients with ≥50 kg and ≤100 kg body weight.

Generally, a body weight adjusted dosing is possible and in a further aspect preferred.

It is also preferred to administer the compounds of the invention in age appropriate formulations. In particular, pediatric administration forms for patients 2 years require a specific administration form, examples include sirups, solutions, drops or formulations for dissolution in a liquid drink.

In a further aspect it is possible to select one of the above defined dosages as an initial dose and subsequently administer 1 or more times the same or varying doses of those defined above in repeating intervals of 1 to 7 days, 1 to 5 days, preferably of 1 to 3 days, or every second day.

The initial dose and the subsequent doses can be selected among the above defined dosages and adjusted/varied in accordance with the need of the patient within the provided ranges.

In particular, the amount of subsequent doses can be appropriately selected depending on the individual patient, the course of disease and the treatment response. It is possible to administer 1, 2, 3, 4, 5, 6, 7, and more subsequent doses.

It is possible that the initial dose is equal or different to the one or more subsequent doses. It is further possible, that the subsequent doses are equal or different.

The repeating intervals can be of the same length or can be varied depending on the individual patient, the course of disease and the treatment response.

Preferably, the subsequent doses are of decreasing amount with increasing number of subsequent dosing.

Preferably a dose of between 3 mg and 300 mg, more preferred between 5 mg and 250 mg, most preferred of 5 mg, 15 mg, 30 mg, 60 mg, 90 mg, 120 mg or 240 mg is administered once daily over a treatment period of at least 3 days, at least 5 days, at least 7 days, up to 4 weeks. In a further preferred aspect a dose of 30 mg, 60 mg or 120 mg is administered once daily. In a further preferred aspect a total daily dose of 30 mg, 60 mg or 120 mg is administered by administering twice daily a 15 mg, 30 mg or 60 mg dose, respectively.

In a further aspect a total daily dose of 240 mg is administered by administering twice daily a 120 mg dose.

In a further aspect, varying doses are administerd by starting with an initial dose, selected among the doses defined above, for 1 week, 2 weeks, 3 weeks, 4 weeks or more, followed by administering an increased dose, selected among the doses defined above, for an additional time period of 1 week, 2 weeks, 3 weeks, 4 weeks or more. Depending on the treatment results, it is also possible to start with a higher initial dose for a time period defined above, followed by a reduced dose for a subsequent treatment interval. Preferably, an initial daily dose of 30 mg or 60 mg is administered for 4 weeks, followed by administering the same dose for further 4 weeks or followed by administering an increased dose for further 4 weeks. Such a treatment regimen may comprise administering 30 mg daily for 4 weeks, followed by administering 60 mg daily for additional 4 weeks.

In particular, doses up to a total daily dose of 240 mg turned out to be safe and well tolerated. The preferred dosing regimen further showed fast oral absorption with detectable levels as early as 15 to 30 minutes post-dose. The absorption level can be maintained stable even upon repeated dosing and no critical accumulation is observed.

The preferred dosing regimen further turned out to efficiently decrease mean serum iron levels and mean calculated transferrin saturation indicating its efficiency for treating SCD.

### Combination Therapies

A further object of the present invention relates to medicaments or combined preparations containing the compound of the present invention and at least one further pharmaceutically active compound (“combination therapy compound”), preferably an additional active compound being useful in the treatment of SCD. Combination therapy compounds may be selected from active compounds used in the prophylaxis and treatment of iron overload and the associated symptoms, including iron-chelating compounds, or compounds for the prophylaxis and treatment of any of the states, disorders or diseases accompanying or resulting from iron overload. Suitable combination therapy compounds may be selected from pharmaceutically active compounds for the prophylaxis and treatment of SCD, thalassemia, haemochromatosis, neurodegenerative diseases (such as Alzheimer's disease or Parkinson's disease) and the associated symptoms. Preferably, the at least one additional pharmaceutically active combination therapy compound is selected from drugs for treating SCD, such as Hydroxyurea, Voxelotor, ADAKVEO® (crizanlizumab), L-glutamine oral powder (Endari®), fetal hemoglobin (HbF) inducers, inhibitors of PDE9 (such as IMR-687), and/or pain-relieving medications. The most preferred combination therapy compound from the group of drugs for treating SCD is fetal hemoglobin (HbF) inducers.

The at least one additional pharmaceutically active combination therapy compound may further be selected from drugs for reducing iron overload (e.g. Tmprss6-ASO) and iron chelators, in particular curcumin, SSP-004184, Deferitrin, deferasirox, deferoxamine and deferiprone as well as JAK2 inhibitors. The most preferred combination therapy compound from the group of iron-chelating compounds is deferasirox.

Further preferred combination therapy compounds may be selected from drugs for treating β-thalassemia, such as Luspatercept®, LentiGlobin® BB305 (a gene therapy developed by the company Bluebird Bio synthetic human hepcidin (LJPC-401), the hepcidin peptidomimetic PTG-300 and the anti-sense oligonucleotide targeting Tmprss6 (IONIS-TMPRSS6-L RX).

In a further aspect the present invention relates to the new use and medical treatment as defined herein, wherein the compounds as defined herein are administered to the patient in need thereof in a combination therapy with one or more of the combination therapy compounds defined above in a fixed dose or free dose combination for sequential use. Such a combination therapy comprises co-administration of the compounds as defined in the present invention with the at least one additional pharmaceutically active compound (drug/combination therapy compound).

Combination therapy in a fixed dose combination therapy comprises co-administration of the compounds as defined herein with the at least one additional pharmaceutically active compound in a fixed-dose formulation.

Combination therapy in a free dose combination therapy comprises co-administration of the compounds as defined herein and the at least one additional pharmaceutically active compound in free doses of the respective compounds, either by simultaneous administration of the individual compounds or by sequential use of the individual compounds distributed over a time period.

In a particularly preferred embodiment, a combination therapy comprises concurrent oral administration of the Compound No. 127 and a combination therapy compound from the group of SCD medicaments, preferably of Hydroxyurea and/or pain-relieving drugs.

A further embodiment of the present invention relates to a combination therapy as described herein, wherein the drug compound is one selected among those described in WO2020/123850 A1, in particular one of the particular example compounds thereof as described below.

A further aspect relates to providing a new combination therapy for treating SCD by administering Compound 127, as described herein, in a combination therapy with Hydroxyurea, Voxelotor, ADAKVEO® (crizanlizumab), L-glutamine oral powder (Endari®), fetal hemoglobin (HbF) inducers, inhibitors of PDE9 (such as IMR-687), and/or pain-relieving medications. The most preferred combination therapy compound from the group of drugs for treating SCD in combination with Compound 127, is fetal hemoglobin (HbF) inducers.

### Further Useful Compounds

Other useful compounds for treating SCD are described in WO2017/068089, WO2017068090A1 and WO 2018/192973. Thus, in some embodiments, a patient is treated by administering a compound of Formula (I)

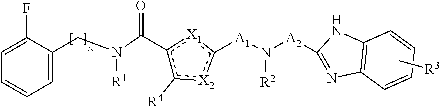

wherein

### X¹ is N or O; and

### X² is N, S or O;

with the provison that X¹ and X² are different;

R¹ is selected from the group consisting of
hydrogen and
optionally substituted alkyl;
n is an integer of 1 to 3;
A¹ and A² are independently selected from the group of alkanediyl;
hydrogen, or
optionally substituted alkyl;
or
A¹ and R² together with the nitrogen atom to which they are bonded form an optionally substituted 4- to 6-membered ring;
R³ indicates 1 , 2 or 3 optional substituents, which may independently be selected from the group consisting of:
halogen,
cyano,
optionally substituted alkyl,
optionally substituted alkoxy, and
a carboxyl group;
R⁴ is selected from the group consisting of
hydrogen,
halogen,
C1-C3-alkyl, and
halogen substituted alkyl.
In some embodiments:
n=1;
R²=hydrogen;
R³=hydrogen;
R⁴=hydrogen;
A¹=methylene or ethane-1,2-diyl;
A²=methylene, ethane-1,2-diyl or propane- 1,3-diyl;
or A¹ and R² together with the nitrogen atom to which they are bonded form an optionally substituted 4-membered ring, forming compounds according to formula (II) or fomula (III)

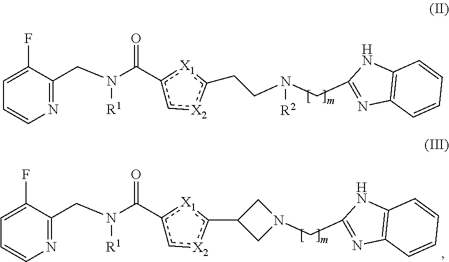

wherein in formula (II) and (III)
m is an integer of 1 , 2 or 3 and
X¹ , X², and R¹ have the meaning as defined for compounds according to formula (I).
In some embodiments the patient is treated with a compound selected from the group consisting of:

| Exp | |
|---|---|
| No. | Structure |
| 1 | 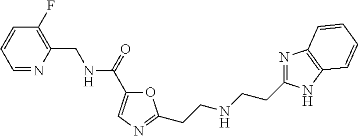 [SMILES: O=C(NCc1ncccc1F)c1cnc(CCNCCc2nc3ccccc3[nH]2)o1] |
| 2 | 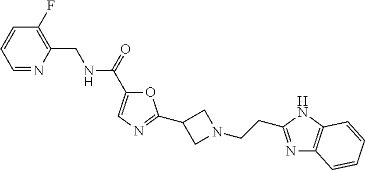 [SMILES: O=C(NCc1ncccc1F)c1cnc(C2CN(CCc3nc4ccccc4[nH]3)C2)o1] |
| 4 | 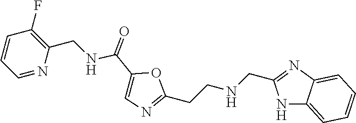 [SMILES: O=C(NCc1ncccc1F)c1cnc(CCNCc2nc3ccccc3[nH]2)o1] |
| 40 | 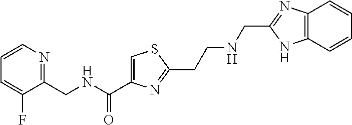 [SMILES: O=C(NCc1ncccc1F)c1csc(CCNCc2nc3ccccc3[nH]2)n1] |
| 94 | 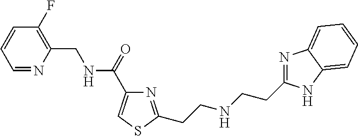 [SMILES: O=C(NCc1ncccc1F)c1csc(CCNCCc2nc3ccccc3[nH]2)n1] |
| 118 | 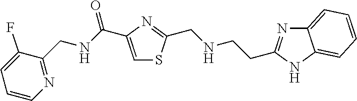 [SMILES: O=C(NCc1ncccc1F)c1csc(CNCCc2nc3ccccc3[nH]2)n1] |
| 126 | 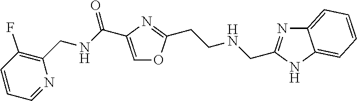 [SMILES: O=C(NCc1ncccc1F)c1coc(CCNCc2nc3ccccc3[nH]2)n1] |
| 127 | 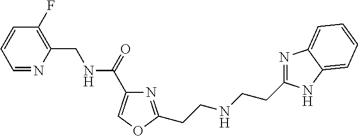 [SMILES: O=C(NCc1ncccc1F)c1coc(CCNCCc2nc3ccccc3[nH]2)n1] |
| 193 | 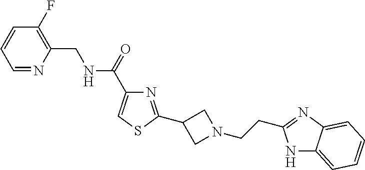 [SMILES: O=C(NCc1ncccc1F)c1csc(C2CN(CCc3nc4ccccc4[nH]3)C2)n1] |
| 206 | 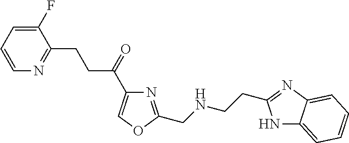 [SMILES: O=C(CCc1ncccc1F)c1coc(CNCCc2nc3ccccc3[nH]2)n1] |
| 208 | 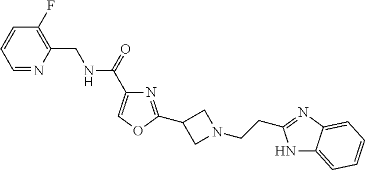 [SMILES: O=C(NCc1ncccc1F)c1coc(C2CN(CCc3nc4ccccc4[nH]3)C2)n1] |
| 233 | 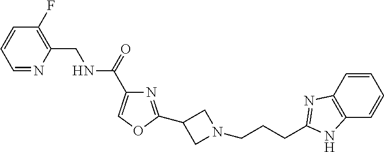 [SMILES: O=C(NCc1ncccc1F)c1coc(C2CN(CCCc3nc4ccccc4[nH]3)C2)n1] |

and pharmaceutically acceptable salts thereof.
In a further preferred aspect the present invention relates to the new use and method of treatment as defined herein, wherein the compounds of the formulae (I), (II) or (III) are selected from the group consisting of:

| Exp. | |
|---|---|
| No. | Structure |
| 1 | 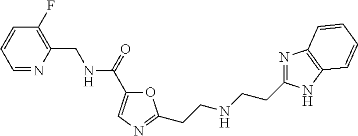 [SMILES: O=C(NCc1ncccc1F)c1cnc(CCNCCc2nc3ccccc3[nH]2)o1] |
| 2 | 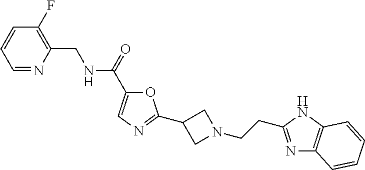 [SMILES: O=C(NCc1ncccc1F)c1cnc(C2CN(CCc3nc4ccccc4[nH]3)C2)o1] |
| 4 | 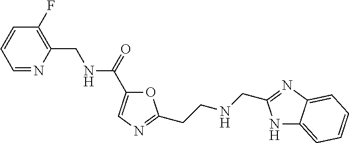 [SMILES: O=C(NCc1ncccc1F)c1cnc(CCNCc2nc3ccccc3[nH]2)o1] |
| 126 | 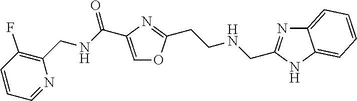 [SMILES: O=C(NCc1ncccc1F)c1coc(CCNCc2nc3ccccc3[nH]2)n1] |
| 127 | 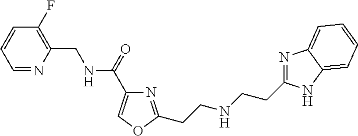 [SMILES: O=C(NCc1ncccc1F)c1coc(CCNCCc2nc3ccccc3[nH]2)n1] |
| 206 | 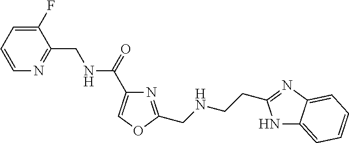 [SMILES: O=C(NCc1ncccc1F)c1coc(CNCCc2nc3ccccc3[nH]2)n1] |
| 208 | 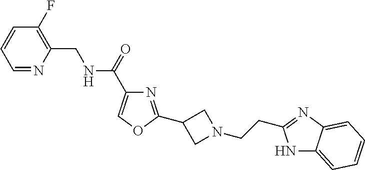 [SMILES: O=C(NCc1ncccc1F)c1coc(C2CN(CCc3nc4ccccc4[nH]3)C2)n1] |
| 233 | 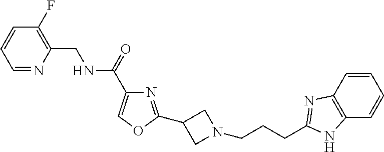 [SMILES: O=C(NCc1ncccc1F)c1coc(C2CN(CCCc3nc4ccccc4[nH]3)C2)n1] |

and pharmaceutically acceptable salts thereof.
In a further preferred aspect the present invention relates to the new use and method of treatment as defined herein, wherein the compounds of the formulae (I), (II) or (III) are selected from the group consisting of:

| Exp. | |
|---|---|
| No. | Structure |
| 1 | 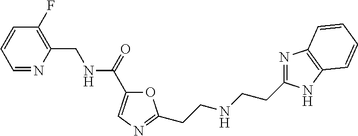 [SMILES: O=C(NCc1ncccc1F)c1cnc(CCNCCc2nc3ccccc3[nH]2)o1] |
| 127 | 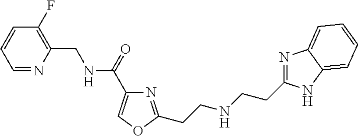 [SMILES: O=C(NCc1ncccc1F)c1coc(CCNCCc2nc3ccccc3[nH]2)n1] |
| 208 | 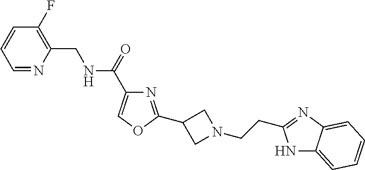 [SMILES: O=C(NCc1ncccc1F)c1coc(C2CN(CCc3nc4ccccc4[nH]3)C2)n1] |

and pharmaceutically acceptable salts thereof.
In some embodiments the method comprises adminstering a compound selected from

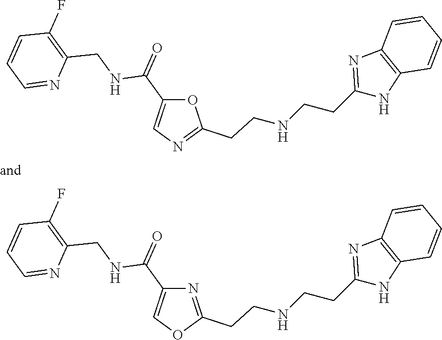 [SMILES: O=C(NCc1ncccc1F)c1cnc(CCNCCc2nc3ccccc3[nH]2)o1 ; O=C(NCc1ncccc1F)c1coc(CCNCCc2nc3ccccc3[nH]2)n1]

and pharmaceutically acceptable salts thereof.

In Formulas I, II and III the substituent groups are defined as follows:

Optionally substituted alkyl preferably includes: linear or branched alkyl preferably containing 1 to 8, more preferably 1 to 6, particularly preferably 1 to 4, even more preferred 1, 2 or 3 carbon atoms, also being indicated as C₁—C₄-alkyl or C₁—C₃-alkyl.

Optionally substituted alkyl further includes cycloalkyl containing preferably 3 to 8, more preferably 5 or 6 carbon atoms.

Examples of alkyl residues containing 1 to 8 carbon atoms include: a methyl group, an ethyl group, an n-propyl group, an i-propyl group, an n-butyl group, an i-butyl group, a sec-butyl group, a t-butyl group, an n-pentyl group, an i-pentyl group, a sec-pentyl group, a t-pentyl group, a 2-methylbutyl group, an n-hexyl group, a 1-methylpentyl group, a 2-methylpentyl group, a 3-methylpentyl group, a 4- methylpentyl group, a 1-ethylbutyl group, a 2-ethylbutyl group, a 3-ethylbutyl group, a 1 , 1 -dimethyl butyl group, a 2,2-dimethylbutyl group, a 3,3-dimethylbutyl group, a 1-ethyl-1-methylpropyl group, an n-heptyl group, a 1-methylhexyl group, a 2-methylhexyl group, a 3-methylhexyl group, a 4-methylhexyl group, a 5-methylhexyl group, a 1-ethylpentyl group, a 2-ethylpentyl group, a 3-ethylpentyl group, a 4-ethylpentyl group, a 1,1-dimethylpentyl group, a 2,2-dimethylpentyl group, a 3,3-dimethylpentyl group, a 4,4-dimethylpentyl group, a 1-propylbutyl group, an n-octyl group, a 1-methylheptyl group, a 2-methylheptyl group, a 3-methylheptyl group, a 4-methylheptyl group, a 5-methylheptyl group, a 6-methylheptyl group, a 1-ethylhexyl group, a 2-ethylhexyl group, a 3-ethylhexyl group, a 4-ethylhexyl group, a 5-ethylhexyl group, a 1,1-dimethylhexyl group, a 2,2-dimethylhexyl group, a 3,3-dimethylhexyl group, a 4,4-dimethylhexyl group, a 5,5-dimethylhexyl group, a 1-propyl pentyl group, a 2-propylpentyl group, etc. Those containing 1 to 4 carbon atoms (C₁—C₄-alkyl), such as in particular methyl, ethyl, n-propyl, propyl, n-butyl, i-butyl, sec-butyl, and t-butyl are preferred. C₁—C₃-alkyl, in particular, methyl, ethyl, propyl and i-propyl are more preferred. Most preferred are C₁ and C₂ alkyl, such as methyl and ethyl.

Cycloalkyl residues containing 3 to 8 carbon atoms preferably include: a cyclopropyl group, a cyclobutyl group, a cyclopentyl group, a cyclohexyl group, a cycloheptyl group and a cyclooctyl group. A cyclopropyl group, a cyclobutyl group, a cyclopentyl group and a cyclohexyl group are preferred. A cyclopropyl group is particularly preferred.

Substituents of the above-defined optionally substituted alkyl preferably include 1, 2 or 3 of the same or different substituents, selected, for example, from the group consisting of: halogen as defined below, such as preferably F, cycloalkyl as defined above, such as preferably cyclopropyl, optionally substituted heteroaryl as defined below, such as preferably a benzimidazolyl group, optionally substituted amino as defined below, such as preferably an amino group or benzyloxycarbonylamino, a carboxyl group, an aminocarbonyl group as defined below, as well as an alkylene group such as in particular a methylene-group, forming for example a methylene-substituted ethyl-group (CH₃—(C═CH₂)—or

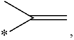 [SMILES: *C(=C)C]

wherein * indicates the binding site).

Halogen includes fluorine, chlorine, bromine and iodine, preferably fluorine or chlorine, most preferred is fluorine.

Examples of a linear or branched alkyl residue substituted by halogen and containing 1 to 8 carbon atoms include: a fluoromethyl group, a difluoromethyl group, a trifluoromethyl group, a chloromethyl group, a dichloromethyl group, a trichloromethyl group, a bromomethyl group, a dibromomethyl group, a tribromomethyl group, a 1-fluoroethyl group, a 1-chloroethyl group, a 1-bromoethyl group, a 2-fluoroethyl group, a 2-chloroethyl group, a 2-bromoethyl group, a difluoroethyl group such as a 1,2-difluoroethyl group, a 1,2-dichloroethyl group, a 1,2-dibromoethyl group, a 2,2-difluoroethyl group, a 2,2- dichloroethyl group, a 2,2-dibromoethyl group a 2,2,2-trifluoroethyl group, a heptafluoroethyl group, a 1-fluoropropyl group, a 1-chloropropyl group, a 1-bromopropyl group, a 2-fluoropropyl group, a 2-chloropropyl group, a 2-bromopropyl group, a 3-fluoropropyl group, a 3-chloropropyl group, a 3-bromopropyl group, a 1,2-difluoropropyl group, a 1,2-dichloropropyl group, a 1,2-dibromopropyl group, a 2,3-difluoropropyl group, a 2,3-dichloropropyl group, a 2,3-dibromopropyl group, a 3,3,3-trifluoropropyl group, a 2,2,3,3,3-pentafluoropropyl group, a 2-fluorobutyl group, a 2-chlorobutyl group, a 2-bromobutyl group, a 4-fluorobutyl group, a 4-chlorobutyl group, a 4-bromobutyl group, a 4,4,4-trifluorobutyl group, a 2,2,3,3,4,4,4-heptafluorobutyl group, a perfluorobutyl group, a 2-fluoropentyl group, a 2-chloropentyl group, a 2-bromopentyl group, a 5-fluoropentyl group, a 5-chloropentyl group, a 5-bromopentyl group, a perfluoropentyl group, a 2-fluorohexyl group, a 2-chlorohexyl group, a 2-bromohexyl group, a 6-fluorohexyl group, a 6-chlorohexyl group, a 6-bromohexyl group, a perfluorohexyl group, a 2-fluoroheptyl group, a 2-chloroheptyl group, a 2-bromoheptoyl group, a 7-fluoroheptyl group, a 7-chloroheptyl group, a 7-bromoheptyl group, a perfluoroheptyl group, etc. Fluoroalkyl, difluoroalkyl and trifluoroalkyl are mentioned in particular, and trifluoromethyl and mono- and di-fluoroethyl is preferred. Particularly preferred is trifluoromethyl.

Examples of a cycloalkyl-substituted alkyl group include the above-mentioned alkyl residues containing 1 to 3, preferably 1 cycloalkyl group such as, for example: cyclopropylmethyl, cyclobutylmethyl, cyclopentylmethyl cyclohexylmethyl, 2-cyclopropylethyl, 2-cyclobutylethyl, 2-cyclopentylethyl 2-cyclohexylethyl, 2- or 3-cyclopropylpropyl, 2- or 3-cyclobutylpropyl, 2- or 3- cyclopentylpropyl, 2- or 3-cyclohexylpropyl, etc. Preferred is cyclopropylmethyl.

Examples of a heteroaryl-substituted alkyl group include the above-mentioned alkyl residues containing 1 to 3, preferably 1 (optionally substituted) heteroaryl group, such as, for example a pyridinyl, a pyridazinyl, a pyrimidinyl, a pyrazinyl, a pyrazolyl, an imidazolyl, a benzimidazolyl, a thiophenyl, or an oxazolyl group, such as pyridine-2-yl-methyl, pyridine-3-yl-methyl, pyridine-4-yl-methyl, 2-pyridine-2-yl-ethyl, 2-pyridine-1-yl-ethyl, 2-pyridine-3-yl-ethyl, pyridazine-3-yl-methyl, pyrimidine-2-yl-methyl, pyrimidine-4-yl-methyl, pyrazine-2-yl-methyl, pyrazol-3-yl-methyl, pyrazol-4-yl-methyl, pyrazol-5-yl-methyl, imidazole-2-yl-methyl, imidazole-5-yl-methyl, benzimidazol-2-yl-methyl, thiophen-2-yl-methyl, thiophen-3-yl-methyl, 1 ,3-oxazole-2-yl-methyl.

Preferred is an alkyl group which is substituted with a benzimidazolyl group, such as benzimidazol-2-yl-methyl and benzimidazol-2-yl-ethyl.

Examples of an amino-substituted alkyl residue include the above-mentioned alkyl residues containing 1 to 3, preferably 1 (optionally substituted) amino group, as defined below, such as, for example, aminoalkyl (NH₂-alkyl) or mono- or dialkylamino-alkyl, such as aminomethyl, 2-aminoethyl, 2- or 3-aminopropyl, methylaminomethyl, methylaminoethyl, methylaminopropyl, 2-ethylaminomethyl, 3-ethylaminomethyl, 2-ethylaminoethyl, 3-ethylaminoethyl, etc. with 3-aminopropyl being preferred, or an alkyl group, which may be substituted with an optionally substituted alkyloxycarbonylamino group such as a group according to formula

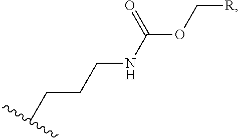

wherein R defines a a phenyl group, forming a benzyloxycarbonylaminopropyl group.

Optionally substituted amino preferably includes: amino (—NH₂), optionally substituted mono- or dialkylamino (alkyl-NH—, (alkyl)₂N—), wherein with respect to “alkyl” reference can be made to the definition of optionally substituted alkyl above. Preferred is mono- or dimethylamino, mono- or diethylamino and monopropylamino. Most preferred is an amino group (—NH₂), and monopropylamino.

Further, a carboxyl group indicates a group [—(C═O)—OH] and an aminocarbonyl group indicates a group [NH₂—(C═O)—].

Optionally substituted alkoxy includes an optionally substituted alkyl-O-group, wherein reference may be made to the foregoing definition of the alkyl group. Preferred alkoxy groups are linear or branched alkoxy groups containing up to 6 carbon atoms such as a methoxy group, an ethoxy group, an n-propyloxy group, an i-propyloxy group, an n-butyloxy group, an i-butyloxy group, a sec-butyloxy group, a t-butyloxy group, an n-pentyloxy group, an i-pentyloxy group, a sec-pentyloxy group, a t-pentyloxy group, a 2-methylbutoxy group, an n-hexyloxy group, an i-hexyloxy group, a t-hexyloxy group, a sec-hexyloxy group, a 2-methylpentyloxy group, a 3-methylpentyloxy group, a 1-ethylbutyloxy group, a 2-ethylbutyloxy group, a 1,1-dimethylbutyloxy group, a 2,2-dimethylbutyloxy group, a 3,3-dimethylbutyloxy group, a 1-ethyl-1-methylpropyloxy group, as well as cycloalkyloxy groups such as a cyclopentyloxy group or a cyclohexyloxy group. A methoxy group, an ethoxy group, an n-propyloxy group and an i-propyloxy group are preferred. A methoxy and ethoxy group is more preferred. Particularly preferred is a methoxy group.

Optionally substituted alkanediyl is preferably a divalent straight-chained or branched alkanediyl radical having from 1 to 6, preferably from 1 to 4, more preferably 1, 2 or 3 carbon atoms, which can optionally carry from 1 to 3, preferably 1 or 2 substituents selected from the group consisting of halogen, hydroxyl (—OH), an oxo group ((═O; forming a carbonyl or acyl group [—(C═O)—]) and an alkyl group as defined above such as preferably methyl. The following may be mentioned as preferred examples: methylene, ethane-1,2-diyl, ethane-1, 1-diyl, propane- 1,3-diyl, propane- 1,1-diyl, propane-1,2-diyl, propane-2,2-diyl, butane-1,4-diyl, butane-1,2-diyl, butane-1,3-diyl, butane-2,3-diyl, butane-1,1-diyl, butane-2,2-diyl, butane-3,3-diyl, pentane-1,5-diyl, etc. Particularly preferred is methylene, ethane-1,2-diyl, ethane-1,1-diyl, propane-1,3-diyl, propane-2,2-diyl, and butane-2,2-diyl. Most preferred are methylene, ethane-1,2-diyl and propane- 1,3-diyl.

A preferred substituted alkanediyl radical is a hydroxy-substituted alkanediyl such as a hydroxy- substituted ethanediyl, an oxo-substituted alkanediyl such as an oxo-substituted methylene or ethanediyl radical, forming a carbonyl or an acyl (acetyl) group, a halogen substituted alkanediyl group.

In some cases, A¹, having the meaning of a linear or branched alkanediyl group as defined above, and R², having the meaning of an optionally substituted alkyl group as defined above, together with the nitrogen atom to which they are bonded form an optionally substituted 4- to 6-membered ring, which may be substituted with 1 to 3 substituents as defined above. Accordingly, A¹ and R² may together from a group according to one the following formulae

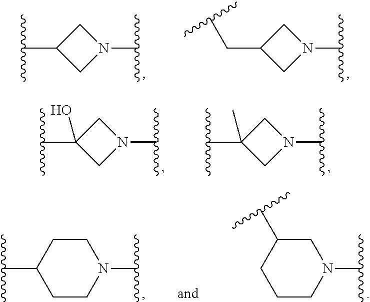

Therein a (substituted or unsubstituted) 4-membered ring- formation is preferred, such as very particularly a group

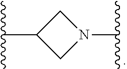 [SMILES: *C1CN(*)C1]

Therein the left-hand binding site indicates the direct binding site to the heterocyclic 5-membered ring between the positions X¹ and X² in formula (I). The right-hand binding site indicates the binding site to the group A2 having the meaning of an alkanediyl group as defined herein.

In the formula (I) as defined herein n has the meaning of an integer of 1 to 3, including 1, 2 or 3 thus indicating a methylene-group, an ethane-1,2-diyl group or a propane-1,3-diyl group. More preferably n is 1 or 2 and even more preferably n is 1, indicating a methylene group.

In some embodiments:

A) X¹ is N or O; and

X² is N, S or O;

with the proviso that X¹ and X² are different;

thus forming 5-membered heterocycles according to the formulae

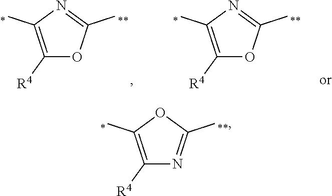

wherein * indicates the binding site to the aminocarbonyl-group and ** indicates the binding site to the A¹-group.

B) n is an integer of 1 , 2 or 3; preferably n is 1 or 2, more preferably n is 1.

C) R¹ is selected from the group consisting of

hydrogen and

optionally substituted alkyl (as defined above);

preferably R¹ is hydrogen or methyl, more preferably R¹ is hydrogen.

D) R² is selected from the group consisting of

hydrogen, and

optionally substituted alkyl (as defined above);

preferably R² is hydrogen or C₁—C₄-alkyl, more preferably R² is hydrogen or methyl, even more preferably R² is hydrogen.

E) R³ indicates 1, 2 or 3 optional substituents, which may independently be selected from the group consisting of

halogen (as defined above),

cyano,

optionally substituted alkyl (as defined above),

optionally substituted alkoxy (as defined above), and

a carboxyl group (as defined above);

preferably R³ indicates 1 or 2 optional substituents, which may independently be selected from the consisting of

halogen,

cyano,

alkyl (as defined above), which may be substituted with 1, 2 or 3 halogen atoms (as defined above),

optionally substituted alkoxy (as defined above), and

a carboxyl group (as defined above);

more preferably R³ indicates 1 or 2 optional substituents, which may independently be selected from the group consisting of

F and CI,

cyano,

trifluoromethyl,

methoxy, and

a carboxyl group;

even more preferably R³ is hydrogen, indicating an unsubstituted terminal benzimidazolyl-ring in formula (I)

F) R⁴ is selected from the group consisting of

hydrogen,

halogen (as defined above),

C₁—C₃-alkyl, and

halogen substituted alkyl (as defined above); preferably R⁴ is selected from the group consisting of

hydrogen

Cl,

methyl, ethyl, iso-propyl, and

trifluoromethyl;

more preferably R⁴ is selected from the group consisting of

hydrogen,

Cl,

methyl, and

trifluoromethyl;

more preferably R⁴ is selected from the group consisting of

hydrogen,

Cl, and

methyl,

even more preferably R⁴ is hydrogen.

G) A¹ is alkanediyl,

preferably A¹ is methylene or ethane-1,2-diyl, more preferably A¹ is ethane-1,2-diyl.

H) A² is alkanediyl,

preferably A² is methylene, ethane-1,2-diyl or propane- 1,3-diyl,

more preferably A² is methylene or ethane-1,2-diyl, even more preferably A² is ethane-1,2-diyl.

or

I) A¹ and R² together with the nitrogen atom to which they are bonded form an optionally substituted 4- to 6-membered ring as defined above;

therein A¹ and R² together with the nitrogen atom to which they are bonded preferably form an optionally substituted 4-membered ring as defined above,

therein A¹ and R² together with the nitrogen atom to which they are bonded more preferably form an unsubstituted 4-membered ring (azetidinyl-ring).

Therein, the substituents of the compounds of the following (I) may in particular have the following meaning:

n has any of the meanings according to B) above and the remaining substituents may have any of the meanings as defined in A) and C) to I).

R¹ has any of the meanings according to C) above and the remaining substituents may have any of the meanings as defined in A) and B) and D) to I).

R² has any of the meanings according to D) above and the remaining substituents may have any of the meanings as defined in A) to C) and E) to H) or I).

R³ has any of the meanings according to E) above and the remaining substituents may have any of the meanings as defined in A) to D) and F) to I).

R⁴ has any of the meanings according to F) above and the remaining substituents may have any of the meanings as defined in A) to E) and G) to I).

A¹ has any of the meanings according to G) above and the remaining substituents may have any of the meanings as defined in A) to F) and H) or I).

A² has any of the meanings according to H) above and the remaining substituents may have any of the meanings as defined in A) to G) and I).

R² and Al have any of the meanings as defined in I) and the remaining substituents may have any of the meanings as defined in A) to C), E), F) and H).

In some cases X¹ is N or O; and X² is N, S or O; with the proviso that X¹ and X² are different; R¹ is hydrogen; n is 1, 2 or 3; A¹ is methylene or ethane-1,2-diyl; A² is methylene, ethane-1,2-diyl or propane-1,3-diyl; R² is hydrogen or C₁—C₄-alkyl;

or

A¹ and R² together with the nitrogen atom to which they are bonded form an optionally substituted 4-membered ring; R³ indicates 1 or 2 optional substituents, which may independently be selected from the group consisting of

halogen,

cyano,

alkyl, which may be substituted with 1, 2 or 3 halogen atoms,

optionally substituted alkoxy, and

a carboxyl group;

R⁴ is selected from the group consisting of

hydrogen

Cl,

methyl, ethyl, iso-propyl, and

trifluoromethyl; or a salt thereof

In some cases the salts are selected from salts of the compounds of formula (I) with acids from the group consisting of benzoic acid, citric acid, fumaric acid, hydrochloric acid, lactic acid, malic acid, maleic acid, methanesulfonic acid, phosphoric acid, succinic acid, sulfuric acid, tartaric acid and toluenesulfonic acid, being characterized by a ratio of compound (I) : acid of 1 to 2 : 1 to 3.

In some cases of the compounds of general Formula I: X¹ is N or O; and X² is N, S or O; with the proviso that X¹ and X² are different; R¹ is hydrogen; n is 1 or 2; A¹ is methylene or ethane-1,2-diyl; A² is methylene, ethane-1,2-diyl or propane-1 ,3-diyl; R² is hydrogen or methyl;

or

A¹ and R² together with the nitrogen atom to which they are bonded form an unsubstituted 4-membered ring;

R³ indicates 1 or 2 optional substituents, which may independently be selected from the group consisting of

F and Cl,

cyano,

trifluoromethyl,

methoxy, and

a carboxyl group;

R⁴ is selected from the group consisting of

hydrogen,

Cl,

methyl, and

trifluoromethyl;

In some cases, the salts are selected from salts of the compounds of formula (I) with acids from the group consisting of benzoic acid, citric acid, fumaric acid, hydrochloric acid, lactic acid, malic acid, maleic acid, methanesulfonic acid, phosphoric acid, succinic acid, sulfuric acid, tartaric acid and toluenesulfonic acid, being characterized by a ratio of compound (I) : acid of 1 to 2:1 to 3.

In some embodiments of the compounds of Formula (I): X¹ is N or O; and X² is N, S or O; with the proviso that X¹ and X² are different; R¹ is hydrogen; n is 1; A¹ is methylene or ethane-1,2-diyl; A² is methylene, ethane-1,2-diyl or propane- 1,3-diyl; R² is hydrogen;

or

A¹ and R² together with the nitrogen atom to which they are bonded form an unsubstituted 4-membered ring; R³ indicates hydrogen, thus forming an unsubstituted terminal benzimidazolyl-ring; R⁴ is selected from the group consisting of

hydrogen,

Cl, and

methyl or a salt thereof;

wherein the salts are selected from salts of the compounds of formula (I) with acids from the group consisting of benzoic acid, citric acid, fumaric acid, hydrochloric acid, lactic acid, malic acid, maleic acid, methanesulfonic acid, phosphoric acid, succinic acid, sulfuric acid, tartaric acid and toluenesulfonic acid, being characterized by a ratio of compound (I) : acid of 1 to 2:1 to 3.

In some embodiments of the compounds of Formula (I): X¹ is N or O; and X² is N, S or O; with the proviso that X¹ and X² are different; R¹ is hydrogen; n is 1; A¹ is methylene or ethane-1,2-diyl; A² is methylene, ethane-1,2-diyl or propane- 1, 3-diyl; R² is hydrogen;

or

A¹ and R² together with the nitrogen atom to which they are bonded form an unsubstituted 4-membered ring; R³ indicates hydrogen, thus forming an unsubstituted terminal benzimidazolyl-ring; and R⁴ is hydrogen; or a salt thereof.

In some embodiments, the salts are selected from salts of the compounds of formula (I), (II), (III) or of the compounds according to WO2020/123850 A1 defined below with acids from the group consisting of benzoic acid, citric acid, fumaric acid, hydrochloric acid, lactic acid, malic acid, maleic acid, methanesulfonic acid, phosphoric acid, succinic acid, sulfuric acid, tartaric acid and toluenesulfonic acid, being characterized by a ratio of compound (I) : acid of 1 to 2:1 to 3.

In some embodiments of the compound of formula (I): n=1; R³ =hydrogen; R⁴=hydrogen; A¹=ethane-1,2-diyl; A² =methylene, ethane-1,2-diyl or propane-1 ,3-diyl; R²=hydrogen; or A¹ and R² together with the nitrogen atom to which they are bonded form an optionally substituted 4-membered ring, forming compounds according to formula (II) or (III) below:

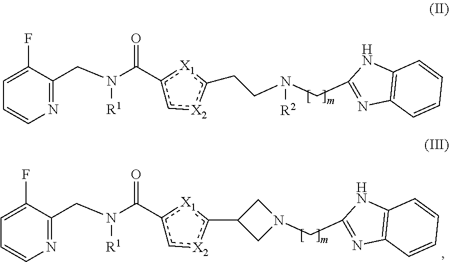

wherein in formula (II) and (III)

m is an integer of 1, 2 or 3 and

X¹, X², and R¹ have the meaning as defined above in any embodiment comprising compounds of formula (I).

In particular, in the formulae (II) and (III) X¹ and X² have the meaning as defined above in A).

In formula (II) R¹ and R² are preferably hydrogen. In formula (III) R¹ is preferably hydrogen and m is preferably 2.

In a further preferred embodiment of the compounds of general formula (II): X¹ and X² are selected from N and O and are different; R¹=hydrogen; R²=hydrogen; and m=2.

Compounds (I), (II) or (III), or the compounds according to WO2020/123850 A1 defined below, forming salts are also referred to as “base” or “free base”. The compounds according to formula (I), (II) or (III), or the compounds according to WO2020/123850 A1 defined below, in the form of the free base have at least one basic group, such as amino groups, to which acidic groups can bind.

The salts of compounds of formula (I), (II) or (III), or of the compounds according to WO2020/123850 A1 defined below, may be selected from salts having a ratio of base (compound (I), (II) or (III)) : acid of 1 to 2:1 to 3, wherein with respect to the salt forming acids reference is made to the selection defined above.

The compounds can mixed salts of a base (compound (I), (II) or (III)) with one or more of the acids indicated above and which may have the same or different ratios base :acid. The acids provide the counter anion for the cationic form of compound (I), (II) or (III).

Particularly preferred are 3HCl salts of the compounds described above.

In a particularly preferred embodiment the method comprises adminstering a 3HCl salt of the Compound 127

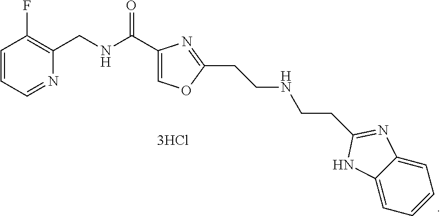 [SMILES: O=C(NCc1ncccc1F)c1coc(CCNCCc2nc3ccccc3[nH]2)n1]

In a further aspect of the invention the Compound 127 may be administered in the form of one of the following salts:

a 1:1 sulfate salt having the formula

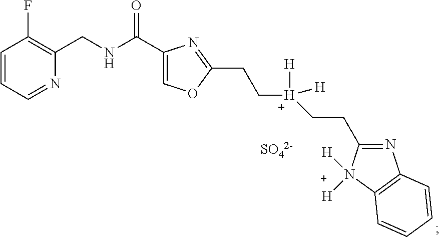

a 1:1 phosphate salt having the formula

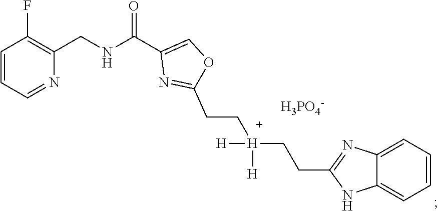

a 2:1 phosphate salt (hemiphosphate)

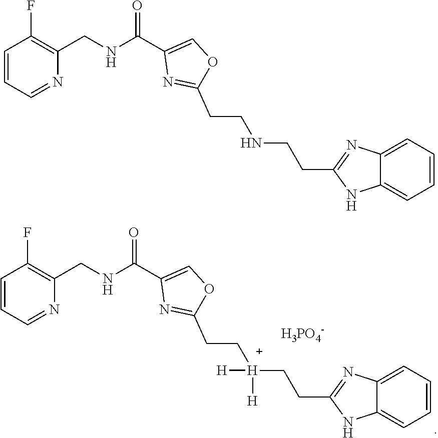

Further compounds acting as ferroportin inhibitors and being suitable in the treatment of SCD as defined herein are those as described in WO2020/123850 A1, incorporated herein by reference in its entirety. Particular compounds among those described in WO2020/123850 A1 being suitable in the treatment of SCD as defined herein can be selected from the group consisting of:

| | | Mass Found |
|---|---|---|
| Structure | IUPAC Name | (M + 1) |
| 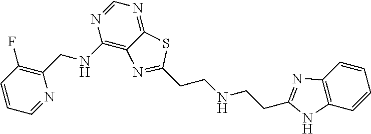 [SMILES: Fc1cccnc1CNc1ncnc2sc(CCNCCc3nc4ccccc4[nH]3)nc12] | 2-(2-{[2-(1H-1,3-benzodiazol- 2-yl)ethyl]amino}ethyl)-N-[(3- fluoropyridin-2-yl)methyl]- [1,3]thiazolo[5,4-d]pyrimidin- 7-amine | 449.2 |
| 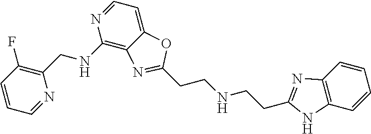 [SMILES: Fc1cccnc1CNc1nccc2oc(CCNCCc3nc4ccccc4[nH]3)nc12] | 2-(2-{[2-(1H-1,3- benzodiazol-2- yl)ethyl]amino}ethyl)-N-[(3- fluoropyridin-2-yl)methyl]- [1,3]oxazolo[4,5-c]pyridin-4- amine | 432.2 |
| 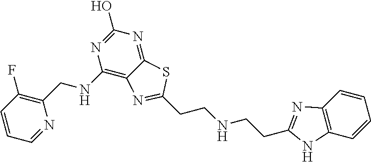 [SMILES: Oc1nc(NCc2ncccc2F)c2nc(CCNCCc3nc4ccccc4[nH]3)sc2n1] | 2-(2-{[2-(1H-1,3- benzodiazol-2- yl)ethyl]amino}ethyl)-7-{[(3- fluoropyridin-2- yl)methyl]amino}- [1,3]thiazolo[5,4-d]pyrimidin- 5-ol | 465.1 |
| 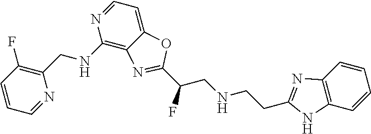 [SMILES: Fc1cccnc1CNc1nccc2oc([C@H](F)CNCCc3nc4ccccc4[nH]3)nc12] | 2-[(1R)-2-{[2-(1H-1,3- benzodiazol-2- yl)ethyl]amino}-1-fluoroethyl]- N-[(3-fluoropyridin-2- yl)methyl]-[1,3]oxazolo[4,5- c]pyridin-4-amine | 450.2 |
| 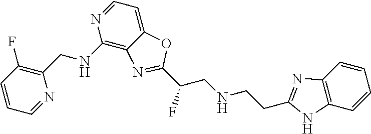 [SMILES: Fc1cccnc1CNc1nccc2oc([C@@H](F)CNCCc3nc4ccccc4[nH]3)nc12] | 2-[(1S)-2-{[2-(1H-1,3- benzodiazol-2- yl]ethyl]amino}-1- fluoroethyl]-N-[(3- fluoropyridin-2-yl)methyl]- [1,3]oxazolo[4,5- c]pyridin-4- amine | 450.2 |
| 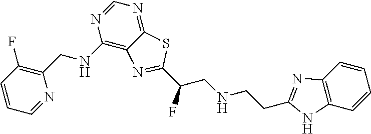 [SMILES: Fc1cccnc1CNc1ncnc2sc([C@H](F)CNCCc3nc4ccccc4[nH]3)nc12] | 2-[(1R)-2-{[2-(1H-1,3- benzodiazol-2- yl)ethyl]amino}-1-fluoroethyl]- N-[(3-fluoropyridin-2- yl)methyl]- [1,3]thiazolo[5,4- d]pyrimidin-7-amine | 467 |

Compounds described in WO2020/123850 A1 and selected from the above group can be provided as a new combination therapy for treating SCD, by administering the compounds in a combination therapy with fetal hemoglobin (HbF) inducers. In a preferred embodiment, the compound 2-(2-{[2-(1H-1,3-benzodiazol-2-yl)ethyl]amino}ethyl)-N-[(3-fluoropyridin-2-yl)methyl]-[1,3]oxazolo [4,5-c]pyridin-4-amine is provided in a combination therapy with a fetal hemoglobin (HbF) inducer for treating SCD.

### DESCRIPTION OF DRAWINGS

FIG. 1: Effect of Compound 127 on hemolysis of RBC from Townes mice. Shown are the plasma levels of cell-free Hb, heme and lactate dehydrogenase (LDH). The hemolysis markers were measured using commercially available kits (cell-free Hb kit # CSB E09632h, Cusabio; heme assays kit #MAK316, Sigma Aldrich), following manufacturer's instructions. LDH was measured in plasma using Hitachi automated clinical chemistry analyzer. Individual values and mean±SD are shown, statistical analysis was performed by comparing all treatment groups to the HbSS vehicle group using one-way ANOVA with Dunnett's multiple comparison test, * p<0.05, ** p<0.01, *** p<0.001, n=9-10 mice per group.

FIG. 2: RBC indices in Townes mice treated for 6 weeks with Compound 127 or vehicle. Individual values and mean±SD are shown, statistical analysis was performed by comparing all treatment groups to the HbSS vehicle group using one-way ANOVA with Dunnett's multiple comparison test, * p<0.05, ** p<0.01, *** p<0.001, n=9-10 mice per group.

FIG. 3: Compound 127 corrected elevated WBC counts in Townes mice. Individual values and mean±SD are shown, statistical analysis was performed by comparing all treatment groups to the HbSS vehicle group using one-way ANOVA with Dunnett's multiple comparison test, * p<0.05, ** p<0.01, *** p<0.001, n=5-10 mice per group.

FIG. 4: Compound 127 reduced spleen and liver size in Townes mice. Individual values and mean±SD are shown, statistical analysis was performed by comparing all treatment groups to the HbSS vehicle group using one-way ANOVA with Dunnett's multiple comparison test, * p<0.05, ** p<0.01, *** p<0.001, n=9-10 mice per group.

FIG. 5: Total and ⁵⁸Fe organ iron levels in Townes mice treated with Compound 127 or vehicle for 6 weeks. Individual values and mean±SD are shown, statistical analysis was performed by comparing all treatment groups to the HbSS vehicle group using one-way ANOVA with Dunnett's multiple comparison test, * p<0.05, ** p<0.01, *** p<0.001, n=8-10 mice per group.

FIG. 6: Compound 127 decreased the percentage of mature RBC containing mitochondria in sickle mice treated with Compound 127 for 6 weeks.

FIG. 7: Compound 127 decreased plasma level of sVCAM-1 in Townes mice. sVCAM-1 was measured by ELISA. Individual values and mean±SD are shown, statistical analysis was performed by comparing all treatment groups to the HbSS vehicle group using one-way ANOVA with Dunnett's multiple comparison test, * p<0.05, ** p<0.01, ***p<0.001, n=6-9 mice per group.

FIG. 8. Plasma iron was determined 3 hours after the last dose of Compound 127 at day 43/44. Individual values with mean±SD are shown. Significant differences to HbSS vehicle group are indicated: * p<0.05, ** p<0.01, *** p<0.001 (One-way ANOVA with Dunnett's multiple comparison test).

FIG. 9. MCHC (left) and CHCM (right) in HbSS and HbAA mice at day 43/44 measured using a Siemens Advia 120 automated blood analyzer. Individual values with mean±SD are shown. Significant differences to HbSS vehicle group are indicated: * p <0.05, ** p<0.01, *** p<0.001 (One-way ANOVA with Dunnett's multiple comparison test).

FIG. 10. Percentage of hypochromic (upper left), microcytic (upper right), hyperchromic (lower left) and macrocytic (lower right) RBCs in male and female HbSS and HbAA at day 43/44 measured using a Siemens Advia 120 automated blood analyzer. Individual values with mean±SD are shown. Significant differences to HbSS vehicle group are indicated: * p<0.05, ** p<0.01, *** p<0.001 (One-way ANOVA with Dunnett's multiple comparison test).

FIG. 11. Total bilirubin and indirect bilirubin in plasma of HbSS and HbAA mice at study day 43/44. Individual values with mean ±SD are shown. Significant differences to

HbSS vehicle group are indicated: * p<0.05, ** p<0.01, *** p<0.001 (One-way ANOVA with Dunnett's multiple comparison test).

FIG. 12. Plasma levels of sP-selectin (left) and RANTES (right) in plasma of HbSS and HbAA mice at the end of the study. Individual values with mean±SD are shown. Significant differences to HbSS vehicle group are indicated: * p<0.05, ** p<0.01, ***p<0.001 (One-way ANOVA with Dunnett's multiple comparison test).

FIG. 13. Plasma xanthine oxidase (XO) activity and intracellular ROS in whole blood of HbSS and HbAA mice. The plasma activity of XO and the percentage of ROS-positive mature RBCs is shown as individual values with mean ±SD. Significant differences compared to HbSS vehicle group are indicated: * p<0.05, ** p<0.01, *** p <0.001 (One-way ANOVA with Dunnett's multiple comparison test).

FIG. 14. Compound 127 reduced intracellular iron (⁵⁶Fe and ⁵⁸Fe) in RBCs of Townes mice. Intracellular content of ⁵⁶Fe and ⁵⁸Fe was determined in washed RBCs from Townes mice by ICP-MS and normalized to RBC counts. Individual values with mean±SD are shown, statistical analysis was performed by comparing all treatment groups to the HbSS vehicle group using one-way ANOVA with Dunnett's multiple comparison test, * p<0.05, n=6-9 mice per group.

FIG. 15. Compound 127 reduced periportal inflammation and the chemokine CXCL1 mRNA expression in livers of Townes mice. The occurrence of periportal inflammation was evaluated on H&E stained paraffin-sections. mRNA expression of CXCL1 from total liver RNA was evaluated by RT-qPCR. Individual scores or individual deltaCt and mean±SD are shown, statistical analysis was performed by comparing all treatment groups to the HbSS vehicle group using one-way ANOVA with Dunnett's multiple comparison test, * p<0.05, ** p<0.01, ***p<0.001, n=7-11 mice per group.

FIG. 16. Compound 127 reduced a biomarker of liver injury. Alanine transaminase (ALT) activity in plasma was measured using Hitachi automated clinical chemistry analyzer. Individual values with mean±SD are shown, statistical analysis was performed by unpaired, 2-tailed student's t-test, * p<0.05, ** p<0.01, *** p<0.001, n=9-10 mice per group.

FIG. 17. Compound 127 reduced IL-1β mRNA expression in lungs of Townes mice. mRNA expression of IL-1β from total lungs RNA was evaluated by RT-qPCR.

Individual deltaCt and mean±SD are shown, statistical analysis was performed by comparing all treatment groups to the HbSS vehicle group using one-way ANOVA with Dunnett's multiple comparison test, * p<0.05, n=8-12 mice per group.

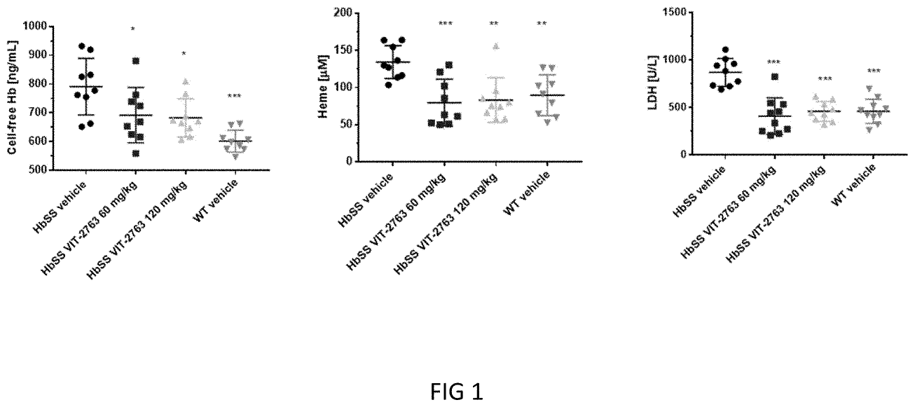

In the FIGS. 1 to 17 “VIT-2763” indicates Compound 127 (in the form of its 3HCl salt).

### DETAILED DESCRIPTION

Townes mouse have been genetically engineered to exclusively express human sickle hemoglobin (Ryan et al. 1990 Science 247:566). Townes mice have anemia, elevated reticulocyte counts, splenomegaly, vascular inflammation and are prone to VO in response to hypoxia, inflammation and hemolysis.

The studies described below used male and female mice homozygous for human HbS (HbSS) and control mice (HbAA) expressing wild-type (WT) human hemoglobin HbA.

### First Study (FIGS. 1 to 7)

The mice were purchased from the Jackson Laboratories (B6;129 Hbbtm2 (HBG1, HBB*)Tow/Hbbtm3(HBG1, HBB)Tow Hbatm1(HBA)Tow/J, Stock Number: 013071; “Townes mice”) at the age of 10 to 12 weeks and were fed a diet with low iron content (10-13 ppm iron, Granovit) and dosed orally twice daily (bid) with Compound 127 at 60 or 120 mg/kg body weight or vehicle (0.5% methylcellulose/water) for 6 weeks excluding weekends. In between compound doses, mice had access to drinking water containing the stable iron isotope ⁵⁸Fe (1 mM ⁵⁸Fe(II)SO₄ supplemented with 10 mM ascorbic acid as a reducing agent) to substitute for the iron present in the standard rodent diets (250 ppm iron). The labeled ⁵⁸Fe allowed to differentiate between iron absorbed during and before the study.

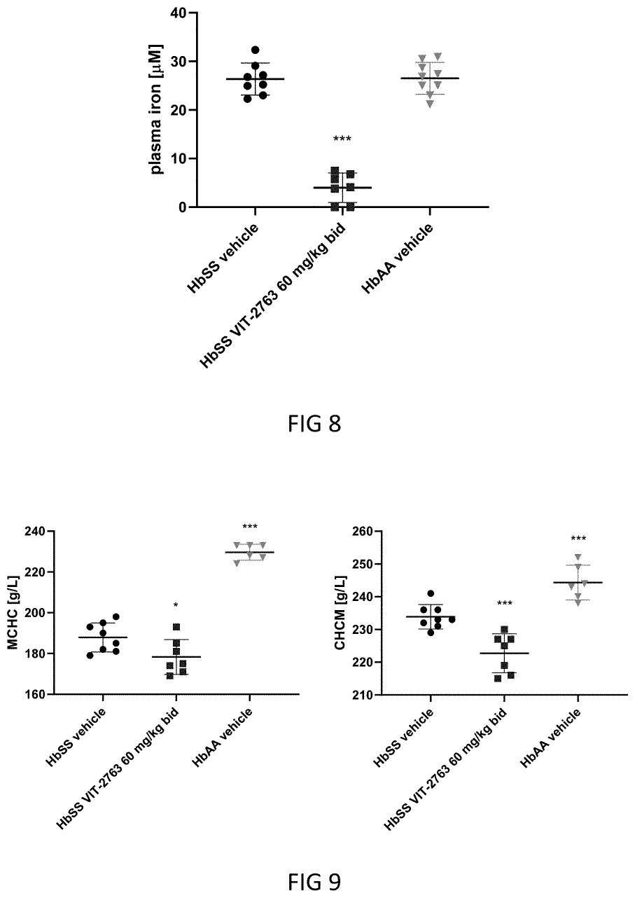

### Second study (FIGS. 8 to 13):

Townes mice (6 weeks of age, Jackson Laboratories, stock#013071) homozygous for HbS (HbSS) were fed a low iron diet (LID, Granovit, Cat. 2039, batch 0001906903, iron content 8.6 mg/kg) and dosed with Compound 127 per os (p.o.) twice daily (bid) at 60 mg/kg or with 0.5% methylcellulose (MC) as a vehicle. Mice had access to drinking water (DW) supplemented with 1 mM ⁵⁸Fe(II) sulfate and 10 mM ascorbic acid for 6 h after the first dose. The concentration of ⁵⁸Fe(II) sulfate supplied in the DW has been adjusted to complement dietary iron supplementation to the level of standard rodent diet with iron content of 250 mg/kg. Water without iron and ascorbic acid was provided during the remaining 18h. Non-sickling Townes mice (HbAA) expressing normal human hemoglobin (wild type, WT) were dosed bid with vehicle and used as controls. Dosing of Compound 127 or vehicle followed by exposure to ⁵⁸Fe-containing water was repeated for 44 days. During the weekends (WE) dosing was paused and mice had access to LID and mineral water without ⁵⁸Fe ad libitum.

Plasma iron was determined using MULTIGENT® Iron assay (Abbott Diagnostics).

Hematological parameters were determined in whole blood samples taken on the last day of the study (day 43/44) and were measured using Siemens Advia ADVIA® 120 System.

ROS in RBCs was detected with the indicator chloromethyl-2′,7′-dichlorodihydrofluorescein diacetate (CM-H2DCFDA, Invitrogen, Cat. C6827) in mature RBCs labelled with APC-eFluor780-conjugated rat anti-mouse Ter119 and PE-conjugated rat anti-mouse CD71 antibodies (eBioscience, Cat. 47-5921-82 and 12-0711).

The activity of the xanthine oxidase in the plasma was measured using the Xanthine Oxidase Activity Assay Kit (Sigma-Aldrich Cat. MAK078).

Plasma bilirubin was measured by assay kit (Sigma-Aldrich, Cat. MAK126), according to the manufacturer's instructions.

Plasma sP-selectin and RANTES were measured by ELISA® kits (R&D Systems, Cat. MVC00 and Cat. DY478-05, resp.), according to the manufacturer's instructions.

The activity of the Compound 127 in prevention of VO for treatment of sickle cell anemia (sickle cell disease) can be determined by using the mouse model described in WO2018/192973, such as e.g. described by Yulin Zhao et al. in “MEK1/2 inhibitors reverse acute vascular occlusion in mouse models of sickle cell disease.” The FASEB Journal Vol. 30, No. 3, pp 1171-1186, 2016. Said mouse model can be suitably adapted to determine the activity of the Compound 127, or of the compounds of further embodiments of the invention, in the treatment of VO in sickle cell anemia. Suitable adaptions to optimized test conditions can be carried out, which is within the routine work of a person skilled in the art.

In the two studies described in Examples 1 to 17, the 3HCl salt of Compound 127 is used.

### EXAMPLE 1: COMPOUND 127 DECREASED HEMOLYSIS IN TOWNES MICE

RBCs of Townes mice are prone to hemolysis as demonstrated by elevated levels of cell-free Hb, heme, LDH in the control HbSS group treated with vehicle (FIG. 1). Notably, Compound 127 decreased significantly the levels of cell-free Hb, heme and LDH, suggesting that ferroportin inhibition by Compound 127 reduced hemolysis in Townes mice (FIG. 1).

### EXAMPLE 2: EFFECT OF COMPOUND 127 ON RBC INDICES

### First Study (FIGS. 1 to 7)

HbSS mice are anemic with pathologically altered hematological parameters indicative for hemolytic anemia, such as reduced RBC counts, Hb and compensatory reticulocytosis and elevated leukocyte counts compared to HbAA mice. Hematologic parameters were measured in fresh EDTA-blood on an automated blood cell analyzer after 6 weeks of treatment with Compound 127. Oral administration of Compound 127 in HbSS mice for six weeks decreased the total Hb, RBC counts, hematocrit, mean corpuscular volume (MCV) and mean corpuscular hemoglobin (MCH). Lowering of the HbS in RBCs of patients with SCD has been associated with decreased HbS aggregation and clinical benefit (Castro O. Am. J. Hematol., 1994).

### Second Study (FIGS. 8 to 13)

Townes HbSS mice have plasma iron levels, similar to the HbAA controls. In the second study, Compound 127 was dosed in HbSS mice for 44 days twice daily at 60 mg/kg and plasma iron was measured 3 h after the last dose of the compound as a marker of acute efficacy. Plasma iron levels in HbSS mice receiving Compound 127 were significantly reduced, demonstrating the efficacy of Compound 127 to inhibit iron transpoprt to blood circulation (FIG. 8).

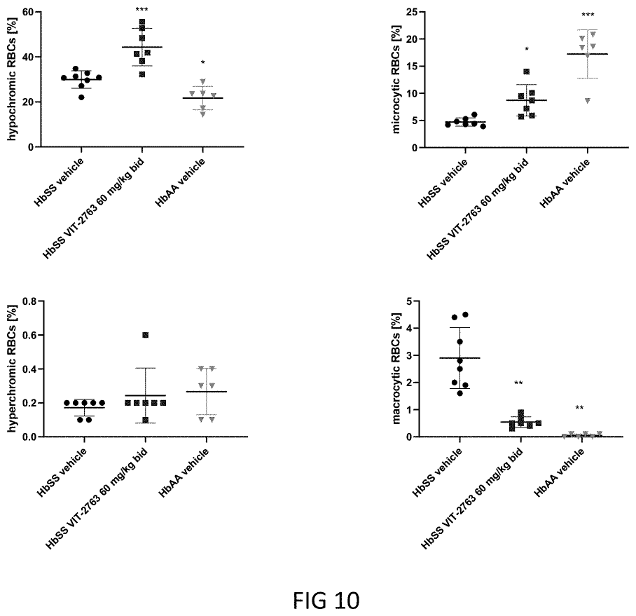

In addition, the mean corpuscular hemoglobin concentration (MCHC) was significantly lowered in Townes mice treated with Compound 127 (FIG. 9, left). MCHC is calculated by dividing the average Hb concentration in lysed blood by the hematocrit. In hemolytic diseases, such as SCD, MCHC value might be incorrectly overestimated due to the presence of free Hb in hemolyzed blood samples. To avoid potential artifacts due to the excessive hemolysis in Townes mice, the concentration of HbS within RBCs was evaluated based on the corpuscular Hb concentration mean (CHCM) parameter. CHCM is measured from laser light scatter and is used to back-calculate a cellular Hb, which reflects the hemoglobin content within intact RBC. CHCM is not affected by hemolysis and was significantly lower in HbSS mice treated with Compound 127, further demonstrating that iron restriction by Compound 127 reduces the concentration of HbS in the SCD model (FIG. 9, right). In addition, scatter plot analysis of the distribution of RBCs based on their volume and Hb concentration revealed a significant increase in the percentage of hypochromic and microcytic RBCs in line with a reduction of macrocytic RBCs in HbSS mice treated with Compound 127 (FIG. 10). The hematological analysis of blood samples from Townes mice indicate, that by blocking ferroportin, Compound 127 is inducing iron restricted erythropoiesis resulting in reduced HbS concentration in RBCs. Lowering the HbS in RBCs of patients with SCD has been associated with decreased HbS aggregation and clinical benefit (Castro O. Am. J. Hematol., 1994). Therefore, the reduction of HbS by Compound 127 might be a novel therapeutic approach in SCD.

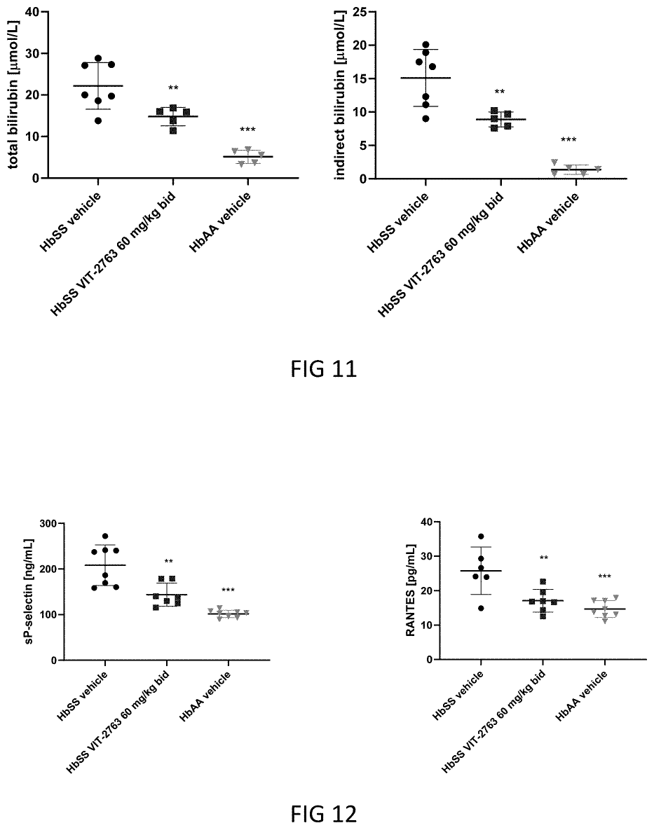

As shown in the first study, Compound 127 attenuated hemolysis in Townes mice, as demonstrated by decreased cell-free Hb, heme and LDH (FIG. 1). In addition, as shown in the second study, Compound 127 lowered the total and indirect bilirubin, which are clinically relevant hemolysis markers, further supporting the efficacy of the compound to reduce hemolysis (FIG. 11).

Interaction of sRBCs, activated leukocytes and free heme with endothelium causes vascular inflammation and promotes vaso-occlusion and organ injury. Endothelial dysfunction in SCD is associated with increased levels of soluble adhesion molecules, such as sVCAM-1 and sP-selectin.

The two independent studies showed, that Compound 127 significantly reduced sVCAM-1 (FIG. 7, first study) and sP-selectin (FIG. 12 left, second independent study), suggesting a potential of Compound 127 to reduce vascular inflammation thereby preventing vaso-occlusion in the Townes model of SCD.

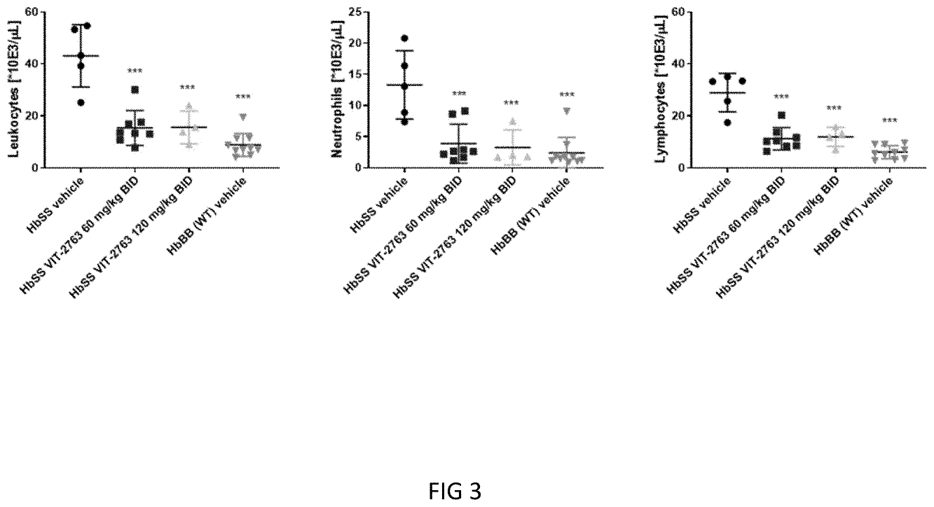

Heme derived from RBCs can act as a damage-associated molecular pattern that activates the innate immune system, leading to oxidant production, inflammation, vaso-occlusion, ischemia, and tissue injury (Belcher JD et al. J. Clin. invest, 2006). Townes mice and SCD patients have elevated leukocytes in the circulation, which produce pro-inflammatory cytokines and chemokines, attracting further inflammatory cells and activating endothelium. As examples, HbSS mice showed increased plasma levels of the chemokine RANTES (CCL5), which is involved in the recruitment of leukocytes to sites of inflammation. Treatment with Compound 127 significantly reduced RANTES levels in HbSS mice (FIG. 12, right), suggesting, that Compound 127 not only decreases the counts of leukocytes in blood circulation (FIG. 3), but also suppresses their pro-inflammatory activity.

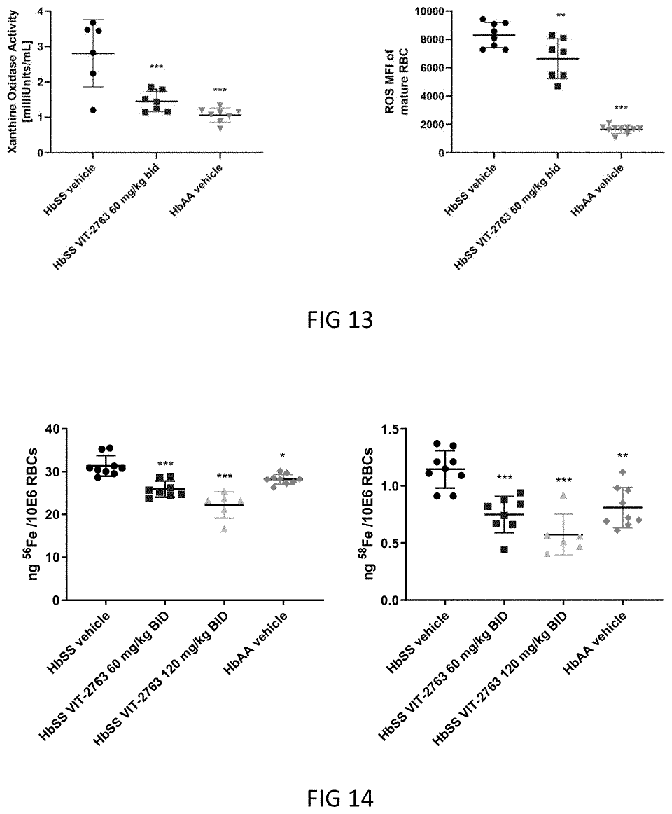

Plasma xanthine oxidase (XO) activity is upregulated in SCD patients and was defined as a source of enhanced vascular superoxide and hydrogen peroxide production. Elevated XO activity is reported also in the plasma of Townes mice (Osarogiagbon UR et al, Blood, 2000; AsIan M et al, PNAS, 2001) and is considered as an important source of ROS production and oxidative tissue damage. Indeed, XO activity was significantly higher in HbSS mice compared to HbAA mice. Compound 127 lowered XO activity in plasma of HbSS mice, suggesting a reduced vascular oxidative damage (FIG. 13 left). Furthermore, flow cytometric analysis of intracellular ROS in blood cells using the fluorescent indicator CM-H2DCFDA showed that the majority of RBCs from HbSS mice have high levels of ROS and treatment with Compound 127 at 60 mg/kg bid lowered them significantly (FIG. 13 right).

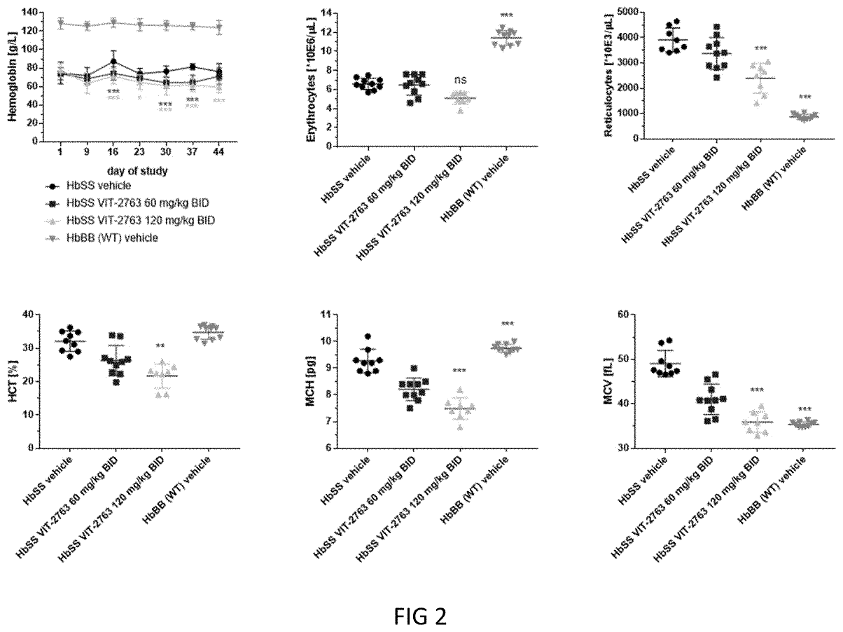

The reduction of HbS by Compound 127 may have positive effects on sRBCs. Importantly, Compound 127 lowered significantly reticulocyte counts, which are greatly expanded in SCD due to compensatory response to hemolysis (FIG. 2).

Together, these data clearly demonstrate the potential of Compound 127 to reduce oxidative stress and vascular inflammation, which might result in decreased adhesion of blood cells to vascular endothelium and ultimately prevention of VO events in the Townes model of SCD.

### EXAMPLE 3: EFFECT OF COMPOUND 127 ON WBC INDICES

Leukocytosis in SCD is associated with increases in the incidence of pain crisis, acute chest syndrome, stroke and mortality (Platt, NEJM, 1991). Unexpectedly, treatment of Townes mice with Compound 127 significantly lowered blood leukocyte counts, particularly neutrophils and lymphocytes (FIG. 3). This data suggests that Compound 127 may have a beneficial effect on inflammation in SCD.

### EXAMPLE 4: COMPOUND 127 REDUCED SPLEEN AND LIVER SIZE IN TOWNES MICE

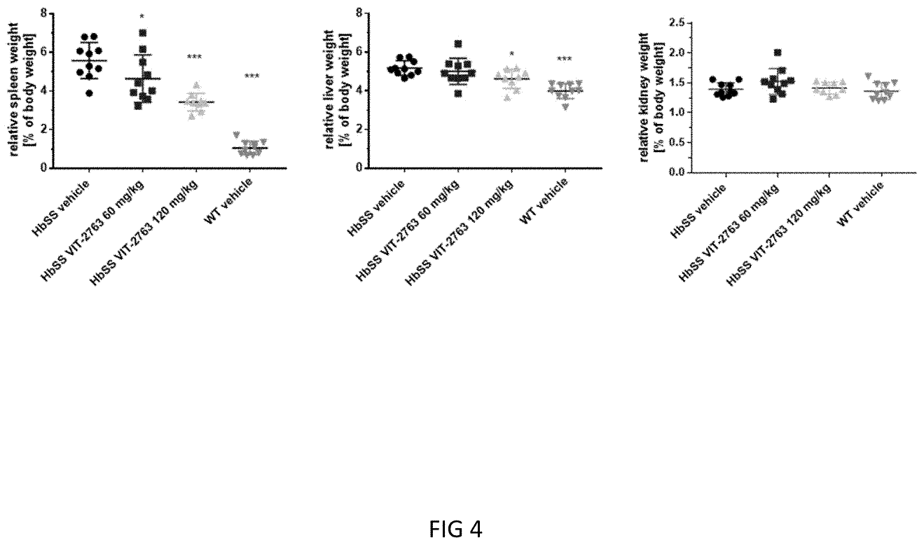

The spleens of Townes mice are greatly enlarged (up to 7-fold larger compared to WT) due to stress erythropoiesis. Notably, Compound 127 reduced the spleen size of Townes mice, demonstrating that Compound 127 improves the extramedullary erythropoiesis (FIG. 4, left). In addition, Compound 127 corrected the increased liver weight of Townes mice to levels close to WT (FIG. 4, middle). The kidney weight in Townes mice is within the range of WT littermates and did not change upon Compound 127 treatment (FIG. 4, right).

### EXAMPLE 5: COMPOUND 127 PREVENTED ORGAN IRON LOADING AND DECREASED THE TOTAL KIDNEY IRON IN TOWNES MICE

Townes mice accumulate excessive iron in organs, such as liver, kidney and spleen as a result of intravascular and extravascular hemolysis of defective RBCs. It is estimated that about ⅓ of hemolysis in SCD is intravascular, due to mechanical destruction of deformed and inelastic sRBCs, while ⅔ is extravascular, resulting from removal of abnormal sRBCs by macrophages (Hebbel RP, Am. J. Hematol., 2011). In addition, anemia results in upregulation of the hypoxia-inducible factor (HIF)-2 alpha in the intestine which causes iron over-absorption (Das N. et al, J.Biol. Chem. 2015).

Oral dosing in rodent PK studies show that Compound 127 is systemically available, suggesting that it is able to block iron export in all ferroportin expressing tissues, including duodenum (dietary iron absorption), liver (iron stores in hepatocytes and macrophages) and spleen (iron in macrophages form senescent erythrocytes). To distinguish the effects of Compound 127 on the pre-existing and newly absorbed iron in organs, mice had access to drinking water containing the stable iron isotope ⁵⁸Fe during the study. Total iron and ⁵⁸Fe content in organs of HbSS mice treated with either vehicle or Compound 127 were analyzed by inductively coupled plasma optical emission spectrometry (ICP-OES) and inductively coupled plasma mass spectrometry (ICP-MS), respectively. Compound 127 did not change the total liver iron concentration and spleen iron content in Townes mice, in agreement with inhibition of ferroportin-mediated iron export from these tissues (FIG. 5, upper left and middle panels). Surprisingly, Compound 127 significantly decreased the total iron concentration in kidneys from Townes mice (FIG. 5, upper right panel). Abnormalities of renal iron metabolism and cortical iron deposition is characteristic for SCD and is involved in renal complications (Vazquez-Meves G et al., Blood, 2016). Therefore, lowering kidney iron by Compound 127 might have beneficial effects on the renal function in SCD.

Importantly, ⁵⁸Fe concentrations in livers, kidneys and spleens of Townes mice dosed with Compound 127 were significantly lower compared to those of vehicle treated mice, indicating that Compound 127 prevents further organ iron accumulation (FIG. 5, lower panels).

### EXAMPLE 6: COMPOUND 127 DECREASED APOPTOSIS MARKERS AND IMPROVED MITOCHONDRIA CLEARANCE IN MATURE sRBCs OF TOWNES MICE

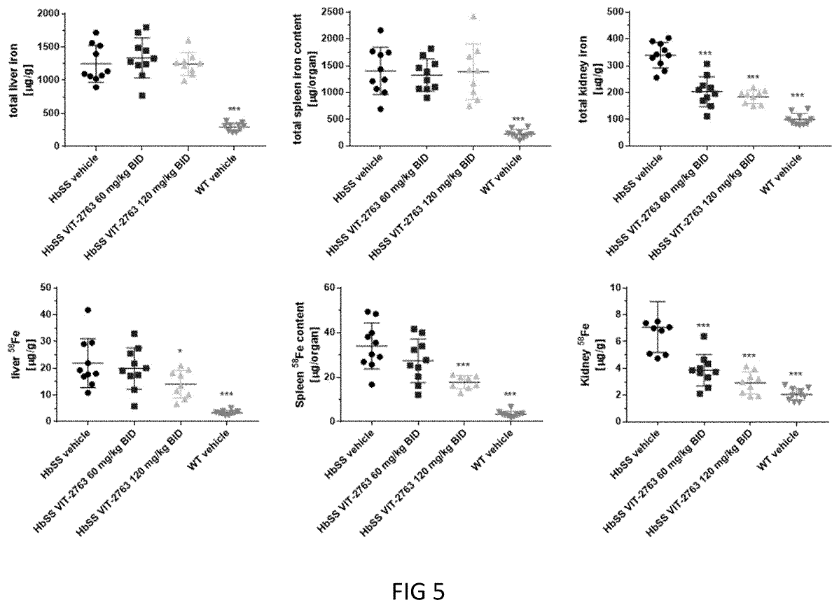

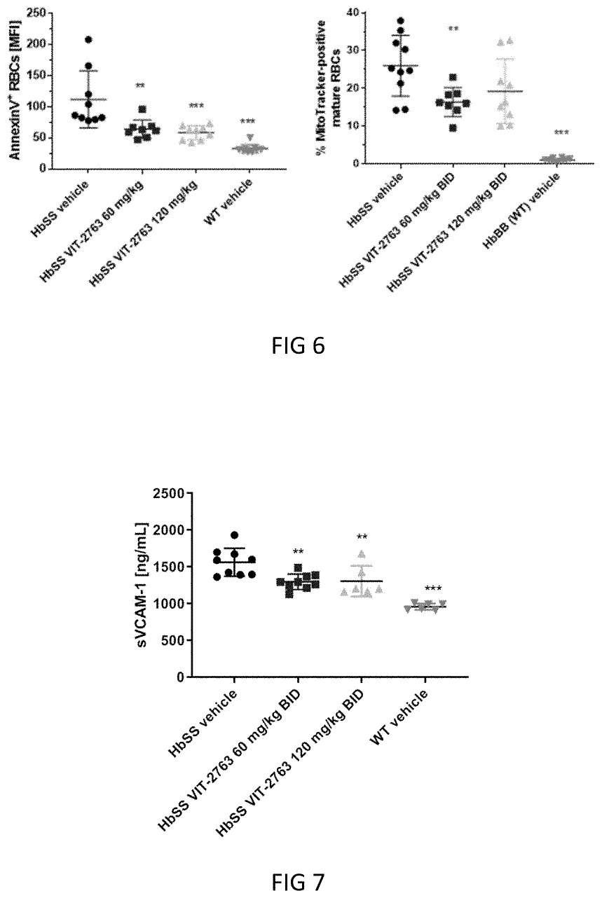

The polymerization of HbS triggers formation of free radicals, dehydration and membrane damage in sRBCs which might lead to cell apoptosis. Apoptotic RBCs expose phosphatidylserine (PS) to the extracellular space which is a signal that targets RBCs for phagocytosis. PS exposure on RBCs was measured by annexin V staining of RBCs and flow cytometry. Compound 127 decreased PS exposure on sRBCs in a dose-dependent manner, indicating that iron restriction improves membrane organization and potentially survival of RBCs (FIG. 6, left).

RBC precursors of healthy individuals eliminate their mitochondria during the terminal differentiation process by mitophagy. Abnormal retention of mitochondria in mature RBCs has been reported for patients and mice with SCD (Jagadeeswaran et al., 2017). Retention of mitochondria in RBCs was studied by flow cytometry using Ter119 and CD71 antibodies to discriminate RBC maturation state and MitoTracker to detect mitochondria. Notably, RBCs of Townes mice receiving Compound 127 at both doses had lower proportion of mature RBCs containing mitochondria (FIG. 6, right). Mouse models with specific deletions in mitophagy genes showed reduced survival of RBCs due to mitochondria retention (Sandoval et al., 2008; Mortensen et al., 2010). This strongly suggests that decreasing the proportion of mitochondria-retaining RBCs by Compound 127 in SCD might positively affect RBCs lifespan.

### EXAMPLE 7: COMPOUND 127 DECREASED VASCULAR INFLAMMATION MARKER IN THE TOWNES MODEL OF SCD

Interaction of sRBCs, activated leukocytes and free heme with endothelium causes vascular inflammation and promotes VO and organ injury. Endothelial dysfunction in SCD is associated with increased levels of soluble adhesion molecules, such as vascular adhesion molecule 1 (sVCAM-1). In agreement with reduced hemolysis (FIG. 1) and reduced leukocyte counts (FIG. 3), treatment with Compound 127 significantly lowered sVCAM-1 levels in Townes mice (FIG. 7). This data demonstrate the potential of Compound 127 to reduce the vascular inflammation in the Townes model of SCD, which might prevent VO events.

### EXAMPLE 8: DETERMINATION OF THE ACTIVITY OF COMPOUND 127 IN THE TREATMENT OF VO IN SICKLE CELL DISEASE IN A MOUSE MODEL

The method as described in WO 2018/192973 can be used to determine the activity of the Ferroportin inhibitor compounds of the present invention.

Using the mouse model as described by Yulin Zhao et al. in “MEK1/2 inhibitors reverse acute vascular occlusion in mouse models of sickle cell disease”; The FASEB Journal Vol. 30, No. 3, pp 1171-1186, 2016, the activity of the Compound 127 in the treatment of sickle cell anemia can be determined as follows:

Vascular occlusion (VO) crises are the major cause of morbidity and mortality in SCD patients. Hypoxia, dehydration, inflammation or hemolysis all contribute to increased adherence of sickle RBCs, neutrophils and platelets to activated endothelium in the small vessels promoting coagulation, vessel obstruction, painful crises and irreversible damage of multiple organs. High leukocyte counts, particularly activated neutrophils, have been correlated with early death, silent brain infarcts, hemorrhagic strokes, and acute chest syndrome in SCD patients (Platt OS, NEJM, 1994). Hemolysis in SCD arises from damaged sickle RBC membranes, causing chronic anemia and the release of Hb into the circulation, which promotes inflammation by depleting NO, generating oxidative stress and releasing heme. Sickle RBCs shed microvesicles which trigger reactive oxygen species (ROS) production by endothelial cells, promote leukocyte adhesion, and induce endothelial apoptosis in a phosphatidylserine-dependent manner, contributing to acute VO in SCD (Camus M, Blood, 2012).

Based on this data, it can be hypothesized that the Compounds of the present invention may alleviate VO in SCD by decreasing hemolysis in sickle RBCs and consecutively preventing leucocyte adhesion to endothelium.

To test this hypothesis vehicle or the Compounds of the present invention are dosed orally at 30 or 100 mg/kg twice daily (BID) for 4 weeks in the Townes mouse model of SCD (Ryan T, Science, 1990). These mice have been genetically engineered to exclusively express human hemoglobin (hα/hα::βS/βS, The Jackson Laboratories). Townes mice have anemia, elevated reticulocyte counts, splenomegaly, vascular inflammation and are prone to VO in response to hypoxia, inflammation and hemolysis. To investigate the effect of Ferroportin inhibitors on leukocyte and sickle RBC adhesion to inflamed endothelium Townes mice treated with vehicle or Ferroportin inhibitor for 25 days are anesthetized and a window chamber is surgically implanted into the dorsal skin fold under sterile conditions, as previously described (Kalambur VS et al., Am J Hematol. 2004; Zennadi, R et al, Blood, 2007). Three days after the surgery mice are injected with 0.5 μg TNFα (R&D Systems) to induce inflammation leading to VO. Ninety minutes after TNFa administration, leukocytes and RBCs are labeled in vivo by intravenous injection of rhodamine-conjugated Ly6G (Sigma) and phycoerythrin-conjugated anti- TER119 mAb (BioLegend), respectively. The adherence of leukocytes and RBS to the endothelium of microvessels is monitored in the following 90 minutes by fluorescent intravital microscopy, as previously described (Zhao et al, FASEB J, 2016). Briefly, anesthetized animals with window chambers are maintained at 37° C., blood flow and cell adhesion events are recorded using a digital video camera C2400 (Hamamatsu Photonics KK, Hamamatsu City, Japan) connected to fluorescent microscope (Axoplan microscope, Carl Zeiss). Twenty to thirty segments of microcapillaries are examined per mouse and cell adherence is quantified on still images by measuring the fluorescence intensity of adherent fluorescence-labeled cells using ImageJ software. Results are expressed as fluorescence units per million cells.

### CONCLUSION

In summary, iron restriction by the oral ferroportin inhibitor Compound 127 significantly reduced hemolysis, oxidative stress, vascular and systemic inflammation and improved RBC morphology, thereby alleviating vaso-occlusive events and improving hemodynamics in the Townes model of SCD.

Ferroportin inhibitors prevent acute vascular occlusion and organ damage in a mouse model of sickle cell disease.

### EXAMPLE 9 (FIG. 14): RBC IRON CONTENT

Sickle RBCs contain several discrete iron compartments, including denatured hemoglobin and free heme, as well as molecular iron associated with membrane phospholipids. Abnormal iron deposits on sickle RBC membranes have been presumed to mediate oxidative damage to membrane structures resulting in their dysfunction (Browne P, Shalev O, Hebbel RP. The molecular pathobiology of cell membrane iron: the sickle red cell as a model. Free Radic Biol Med. 1998 Apr;24(6):1040-8). In addition, high concentrations of HbS (MCHC and CHCM) in sickle RBCs is also associated with cumulative membrane abnormalities, including oxidative damage possibly due to increased membrane iron. Thus, reduction in total intracellular iron content in sickle RBCs might be beneficial in SCD. The intracellular content of ⁵⁶Fe and ⁵⁸Fe was determined in washed RBCs from Townes mice by ICP-MS and normalized to RBC counts. Vehicle-treated HbSS mice had significantly higher levels of both intracellular ⁵⁶Fe and ⁵⁸Fe compared to HbAA mice despite having lower hemoglobin levels suggesting abnormal iron accumlulation. Treatment with Compound 127 normalized intracellular iron content in RBCs of Townes mice. The reduction of intracellular iron content in RBC might not only be linked directly to reduced Hb content (observed as reduced MCHC and CHCM) but also to lower deposition of membrane iron. This result underlines the potential of Compound 127 to minimize the deleterious toxic effects of free iron on RBCs in SCD.

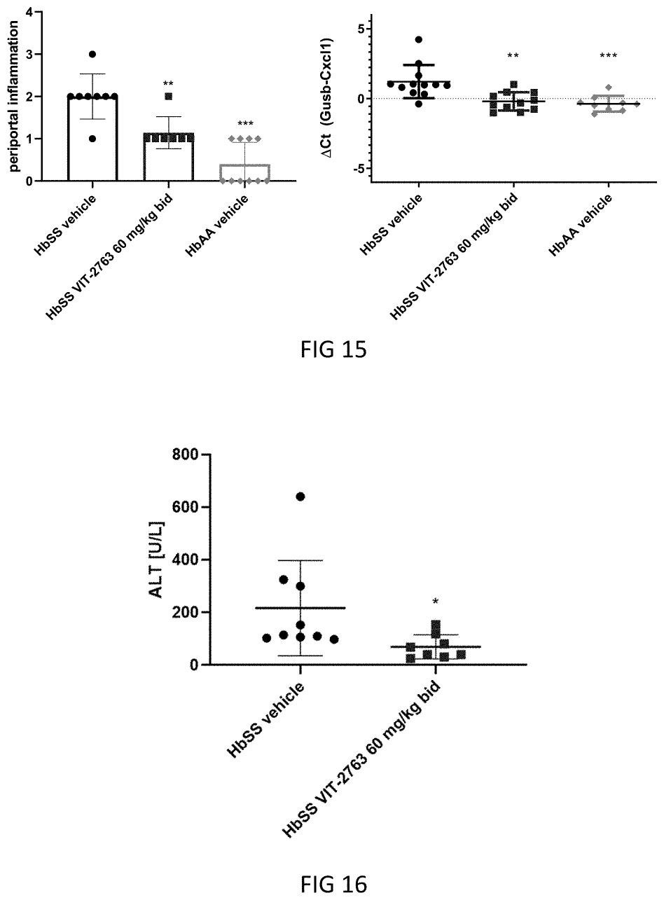

### EXAMPLE 10 (FIG. 15): LIVER INFLAMMATION

Vascular inflammation caused by interactions of sRBC, activated leukocytes and free heme with the endothelium is known to promote vaso-occlusion (VO) and organ damage. Compound 127 significantly reduced markers of vascular inflammation and leukocytes in peripheral blood suggesting a potential to reduce inflammation, VO and consequent organ injury. To assess organ damage, liver lobes isolated from Townes mice treated with Compound 127 for 6 weeks were fixed in formalin and paraffin-sections were stained with hematoxylin and eosin (H&E) for histological examination. Among the main pathological findings were mononuclear inflammatory infiltrates in the periportal regions, which were subsequently scored by a pathologist. The following histological scoring were applied: No inflammation (0), occasional portal tracts show mild to moderate inflammation (1), moderate numbers of portal tracts show mild to moderate inflammation (2), marked inflammation in numerous portal tracts (3). The histopathological evaluation revealed a significant reduction of the occurrence of periportal inflammation in Townes mice treated with Compound 127 compared to vehicle-treated Townes mice further supporting the efficacy of the compound to reduce inflammatory state in SCD.

The chemokine CXCL1 is a critical inflammatory mediator of acute VO crisis in SCD mice (Jang JE. CXCL1 and its receptor, CXCR2, mediate murine sickle cell vaso-occlusion during hemolytic transfusion reactions. J Clin Invest. 2011;121(4):1397-1401). CXCL1 is expressed by activated endothelial cells and is involved in the hepatic recruitment of neutrophils (Hilscher MB. Mechanical Stretch Increases Expression of CXCL1 in Liver Sinusoidal Endothelial Cells to Recruit Neutrophils, Generate Sinusoidal Microthombi, and Promote Portal Hypertension. Gastroenterology. 2019;157(1):193-209), which play important pathological role in SCD crisis. Vehicle-treated Townes mice showed an up-regulation of CXCL1 mRNA expression in the liver compared to vehicle-treated HbAA mice, demonstrating a recruitment of neutrophils in the hepatic tissue, coherently with the histopathological analysis mentioned above. Compound 127 significantly reduced the expression of CXCL1 in the liver of HbSS mice, indicating a protective function against liver inflammation in Townes mice.

### EXAMPLE 11 (FIG. 16): ALT

Liver disease is an important cause of morbidity and mortality in patients with SCD. The Townes mouse model of SCD is known to recapitulate hepatocellular injury which is reflected by elevated plasma alanine transaminase (ALT) levels, a clinically relevant biomarker for liver injury (Aslan M. Oxygen radical inhibition of nitric oxide-dependent vascular function in sickle cell disease. Proc Natl Acad Sci U S A. 2001;98(26):15215-15220). Importantly, Compound 127 significantly lowered plasma ALT levels in Townes mice underlining the potential of the compound to reduce VO tissue injury.

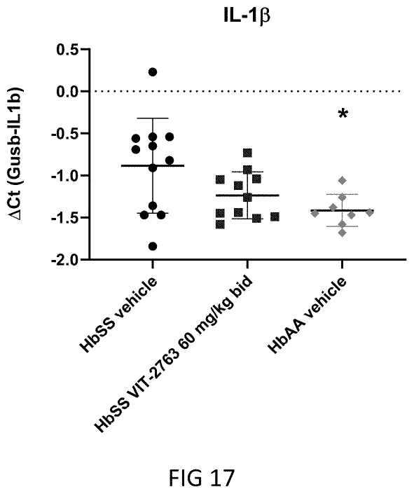

### EXAMPLE 12 (FIG. 17): LUNG INFLAMMATION

Acute chest syndrome (ACS) is a pulmonary complication of SCD patients with a significant overlap with pneumonia. ACS is the second most frequent reason for hospitalization and a leading cause of death in individuals with SCD. VO crisis (VOCs) often precede ACS and are characterized by RBC sickling, cellular hyper-adhesion and hemolysis (Novelli EM. Crises in Sickle Cell Disease. Chest. 2016;149(4):1082-1093). Pro-inflammatory cytokines are elevated in sera of sickle cell patients and are associated with pain crisis and VO. In particular, IL-1β is a pro-inflammatory cytokine released by activated monocytes, able to induce endothelial cells activation and playing a driving role in the pathophysiology of pulmonary microvascular occlusion in SCD (Pathare A. Cytokines in Sickle Cell Disease. Hematology. 2003;8(5):329-337).

Vehicle-treated Townes mice showed an up-regulation of IL-1β mRNA expression in the lungs compared to vehicle-treated HbAA mice. Administration of compound 127 reduced the expression of IL-1β in the lungs of HbSS mice. Although the decrease of IL-1β expression in mice treated with the compound was not significant, mostly due to the high variability in HbSS mice treated with vehicle, the tendency was consistent among independent experiments, suggesting that compound 127 has the potential to reduce lungs inflammation and might protect against pulmonary VO and ACS in SCD.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-15 (canceled)

16. A method of treating sickle cell disease in a human patient, comprising administering to a patient in need thereof, a compound according to the following formula: 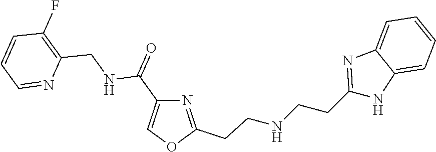 [SMILES: O=C(NCc1ncccc1F)c1coc(CCNCCc2nc3ccccc3[nH]2)n1]
or a pharmaceutically acceptable salt thereof.

17. The method of claim 16, wherein the patient in need thereof exhibits symptoms of at least one of vascular inflammation and vaso-occlusion.

18. The method of claim 17, wherein treating vascular inflammation and vaso-occlusion comprises at least one of treatment of against liver and lung inflammation, reduction of VO tissue injury and protection against pulmonary VO and ACS.

19. The method of claim 16, wherein the pharmaceutically acceptable salt is selected from the group consisting of: benzoic acid salt, HCl salt, citric acid salt, fumaric acid salt, lactic acid salt, malic acid salt, maleic acid salt, methanesulfonic acid salt, phosphoric acid salt, succinic acid salt, sulfuric acid salt, tartaric acid salt and toluenesulfonic acid salt.

20. The method of claim 16, wherein the sickle cell disease is selected from HbSS; HbSC; HbSβ0 thalassemia; HbSβ+ thalassemia, HbSD, HbSE, and HbSO.

21. The method of claim 16, wherein the compound is an HCl salt.

22. The method of claim 16, wherein the compound is a 3HCl salt having the following formula

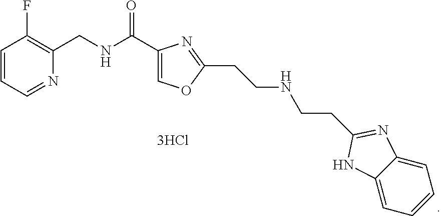 [SMILES: O=C(NCc1ncccc1F)c1coc(CCNCCc2nc3ccccc3[nH]2)n1]

23. The method of claim 20, wherein the compound is an HCl salt.

24. The method of claim 20, wherein the compound is a 3HCl salt having the following formula

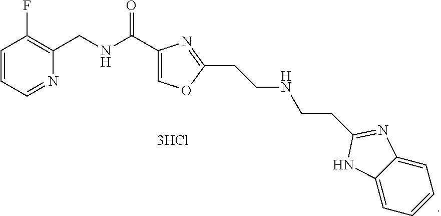 [SMILES: O=C(NCc1ncccc1F)c1coc(CCNCCc2nc3ccccc3[nH]2)n1]

25. The method of claim 16, wherein administering to a patient in need thereof includes oral administration.

26. The method of claim 16, wherein the compound is contained in a filled capsule for oral administration.

27. The method of claim 16, wherein the compound is administered in a daily dose of 5 mg, 15 mg, 30 mg, 60 mg, 120 mg or 240 mg.

28. The method of claim 20, wherein the compound is administered in a daily dose of 5 mg, 15 mg, 30 mg, 60 mg, 120 mg or 240 mg.

29. The method of claim 22, wherein the compound is administered in a daily dose of 5 mg, 15 mg, 30 mg, 60 mg, 120 mg or 240 mg.

30. The method of claim 16, wherein the compound is administered in a daily dose of 30 mg or 60 mg to patients with a body weight of ≥50 kg and ≤100 kg.

31. The method of claim 20, wherein the compound is administered in a daily dose of 30 mg or 60 mg to patients with a body weight of ≥50 kg and ≤100 kg.

32. The method of claim 22, wherein the compound is administered in a daily dose of 30 mg or 60 mg to patients with a body weight of ≥50 kg and ≤100 kg.

33. The method of claim 16, wherein the compound is administered once or twice daily.

34. The method of claim 16, wherein the method forms part of a combination therapy, wherein the combination therapy further comprises co-administration to the patient in need, at least one additional pharmaceutically active compound, and wherein the compound and the at least one additional pharmaceutically active compound are co-administered as a fixed dose or a free dose.

35. The method of claim 34, wherein the at least one additional pharmaceutically active compound is selected from the group of SCD medicaments, Hydroxyurea and pain-relieving drugs.
